(19) United States
(12) Patent Application Publication
(10) Pub. No.: US 2024/0366594 A1
(43) Pub. Date: Nov. 7, 2024

(54) Small Molecule Inhibitors of Formation of Neutrophil-Derived Extracellular Traps (NETosis) and Uses Thereof

(71) Applicants: The Brigham and Women's Hospital, Inc., Boston, MA (US); The General Hospital Corporation, Boston, MA (US); The Children`s Medical Center Corporation, Boston, MA (US)

(72) Inventors: Jeffrey M. Karp, Brookline, MA (US); Ivan Zanoni, Brookline, MA (US); Pui Yan Ma, Boston, MA (US); Ralph Mazitschek, Weston, MA (US); Stephen J. Haggarty, Gloucester, MA (US)

(21) Appl. No.: 18/563,774

(22) PCT Filed: May 27, 2022
(86) PCT No.: PCT/US2022/072609

§ 371 Date: Nov. 22, 2023

Related U.S. Application Data
(60) Provisional application No. 63/194,153, filed on May 27, 2021.

(51) Int. Cl. A61K 31/505; A61K 31/4406; A61K 45/06; A61P 11/00; A61P 37/06; C12Q 1/6883; G01N 33/68

(52) U.S. Cl. CPC A61K 31/505; A61K 31/4406; A61K 45/06; A61P 11/00; A61P 37/06; C12Q 1/6883; G01N 33/6893; C12Q 2600/158

(57) ABSTRACT

Described herein are compositions and methods that inhibit class I and IIb HIDACs for use in inhibiting NETosis and conditions associated with NETosis.

### CLAIM OF PRIORITY

This application claims the benefit of U.S. Provisional Application Ser. No. 63/194, 153, filed on May 27, 2021. The entire contents of the foregoing are incorporated herein by reference.

### TECHNICAL FIELD

Described herein are compositions and methods that inhibit class I and IIb HDACs for use in inhibiting NETosis and conditions associated with NETosis.

### BACKGROUND

The immune system evolved to sense microbial and endogenous cues that alter cellular or organismal homeostasis. When infection and tissue damage coincide, the immune system reacts rapidly to avoid the spread of the infection, even at the cost of an overexuberant reaction that may cause further tissue damage. Neutrophils are key players in this process (Nathan, 2002). These polymorphonuclear cells orchestrate the innate immune response by neutralizing pathogens via several effector mechanisms, which also cause tissue damage (Jaillon et al., 2013). Given their tissue damaging capabilities, neutrophils serve central roles in many inflammatory disorders and favor immune-mediated coagulation and disseminated clot formation (Gomez-Moreno et al., 2018). Central for both pathogen containment as well as development of immune-mediated disorders is the formation of neutrophil-derived extracellular traps (NETs) through a cellular process termed NETosis (Brinkmann et al., 2004; Saitoh et al., 2012; Thiam et al., 2020). Despite the key functions played by NETs against viruses, bacteria, and fungi (Thiam et al., 2020), the dysregulated formation of NETs has been linked to a variety of diseases such as acute respiratory distress syndrome (ARDS) and sepsis. ARDS and sepsis are characterized by an exaggerated elevation of pro-inflammatory cytokines in the lungs and/or bloodstream, and also by coagulopathy, thrombosis, and multiorgan dysfunction (Broggi et al., 2020; Channappanavar and Perlman, 2017; Giannis et al., 2020; Guan et al., 2020; Iba et al., 2019; Lee et al., 2020; Lucas et al., 2020; Mehta et al., 2020; Ruan et al., 2020; Shin et al., 2019; Short et al., 2014; Tang et al., 2019; Wu et al., 2019; Yang et al., 2019; Zhou et al., 2020). The central roles played by neutrophils and NETosis in driving these life-threatening immune disorders provide a strong mandate to reveal new steps in the regulation of NET induction that can be used as novel therapeutic targets.

### SUMMARY

As shown herein, class I and IIb HDACs, which belong to the family of zinc-dependent lysine deacetylases, regulate NETosis in human and mouse cells. Also, these zinc-dependent HDACs can be used as therapeutic targets in mouse models of ARDS and septic shock. In particular, the class I/IIb HDAC inhibitor ricolinostat dampens NET formation and pro-inflammatory cytokine production, reduced morbidity, and improved lung functionality in microbial pneumonia and septic shock.

Thus provided herein are methods for treating a subject who has a condition associated with NETosis, or reducing the risk of developing or delaying onset of a condition associated with NETosis, the methods comprising administering a therapeutically effective amount of an inhibitor of a class I and/or class IIb histone deacetylase (HDAC). Also provided are inhibitors of a class I and/or class IIb histone deacetylase (HDAC) for use in a method for treating a subject who has a condition associated with NETosis, or reducing the risk of developing or delaying onset of a condition associated with NETosis.

In some embodiments, the condition associated with NETosis is an autoimmune disease, a cardiovascular condition, an inflammatory condition, a condition associated with viral or bacterial infection (optionally bacterial-induced pneumonia or conditions associated with COVID-19), cytokine storm, or cancer.

In some embodiments, the autoimmune disease is Antineutrophil cytoplasmic antibody (ANCA)-associated vasculitis (AAV), Type 1 diabetes mellitus (T1DM), Systemic lupus erythematosus (SLE), Rheumatoid Arthritis (RA), psoriasis, Antiphospholipid syndrome (APS), multiple sclerosis (MS), dermatomyositis (DM), polymyositis (PM), and IgG4-related autoimmune pancreatitis (AIP), or Drug-Induced Autoimmune Diseases

In some embodiments, the cardiovascular condition is small vessel vasculitis (SVV) or atherosclerosis.

In some embodiments, the inflammatory conditions is gout or Inflammatory bowel diseases (IBDs).

Also provided herein are methods for treating or reducing the risk of occurrence or reoccurrence, or delaying onset, of a cardiovascular event in a subject who has a condition associated with NETosis. The methods comprise administering a therapeutically effective amount of an inhibitor of a class I/IIb histone deacetylase (HDAC). Also provided are inhibitors of a class I/IIb histone deacetylase (HDAC), for use in a method for treating or reducing the risk of occurrence or reoccurrence, or delaying onset, of a cardiovascular event in a subject who has a condition associated with NETosis. In some embodiments, the cardiovascular event is thrombosis or myocardial infarction (MI).

In some embodiments, of the methods described herein, the subject does not have cancer, e.g., does not have a solid tumor (such as hepatocellular carcinoma) or a hematopoietic cancer, e.g., leukemia or lymphoma, e.g., multiple myeloma, acute myeloid leukemia, or B-cell lymphoma. In some embodiments, the subject does not have diabetic peripheral neuropathy.

In some embodiments, the HDAC inhibitor is an inhibitor of HDAC6.

In some embodiments, the HDAC inhibitor is entinostat, ricolinostat, citarinostat, AES-135; HDAC-IN-3 and analogues thereof; ACY-738; Abexinostat; CAY10603; WT-161; EDO-S101; UF010; Resminostat; HPOB; or CRA-026440.

In some embodiments, the subject has been identified as having a condition associated with NETosis. In some embodiments, the subject has been identified as having a level of circulating cfDNA, MPO/cfDNA conjugates and/or citH3 above a reference level. In some embodiments, the methods comprise obtaining a sample comprising blood from a subject; determining a level of circulating cfDNA, MPO/cfDNA conjugates and/or citH3 in the sample; comparing the level of circulating cfDNA, MPO/cfDNA conjugates and/or citH3 to a reference level that represents a level of circulating cfDNA, MPO/cfDNA conjugates and/or citH3 in a subject who does not have a condition associated with NETosis; and identifying a subject who has a level of circulating cfDNA, MPO/cfDNA conjugates and/or citH3 above a reference level as having a condition associated with NETosis.

In some embodiments, the subject has been identified as having a perforin mutation, as having a serum ferritin level above a threshold or reference level, or as having a level of a damage-associated molecular pattern (DAMP), optionally high mobility group box-1 (HMGB1), above a threshold or reference level.

Also provided herein are compositions comprising (i) an anti-viral drug and (ii) a small molecule inhibitor of class I and/or class IIb HDACs, and optionally further comprising a pharmaceutically acceptable carrier.

In some embodiments, the anti-viral drug is nirmatrelvir and ritonavir; sotrovimab; remdesivir; molnupiravir (Lagevrio), or an antibody, optionally bebtelovimab. In some embodiments, the HDAC inhibitor is entinostat, ricolinostat, citarinostat, AES-135; HDAC-IN-3 and analogues thereof; ACY-738; Abexinostat; CAY10603; WT-161; EDO-S101; UF010; Resminostat; HPOB; or CRA-026440. Also provided are methods of treating a viral infection in a subject, the method comprising administering a therapeutically effective amount of these compositions. In some embodiments, the viral infection is infection with a coronavirus, e.g., SARS-CoV-2.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

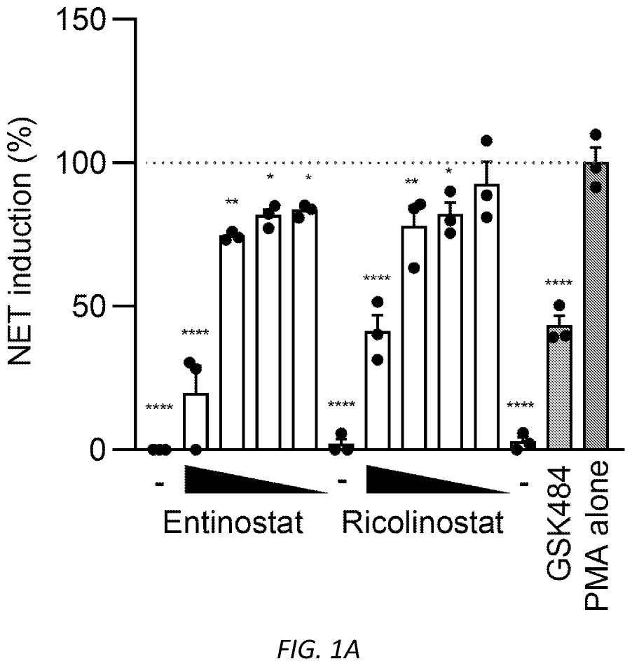

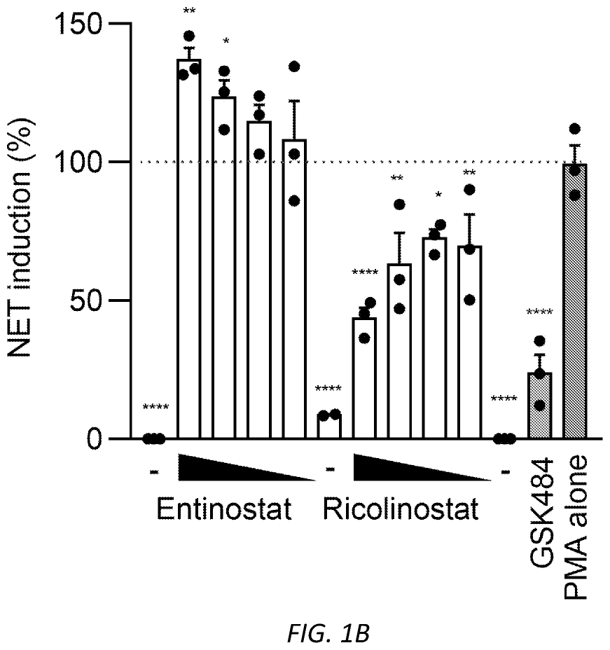

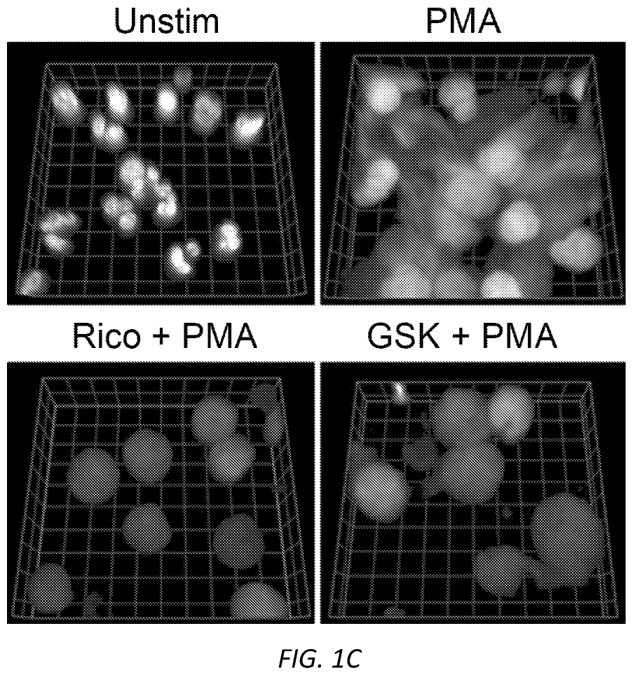

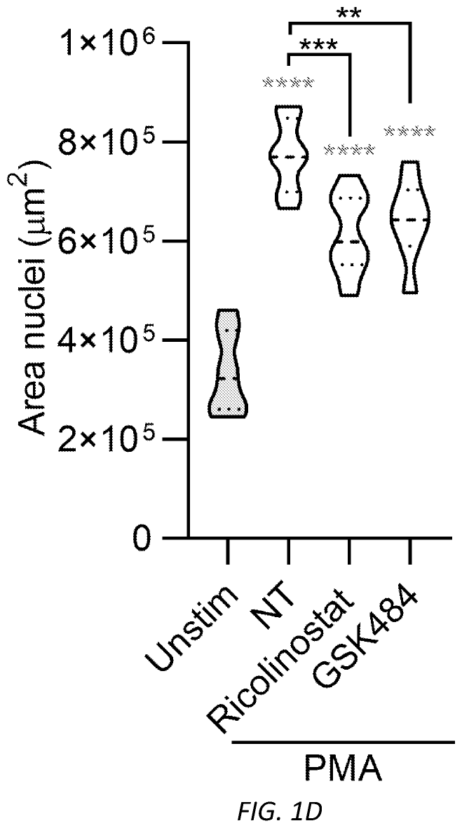

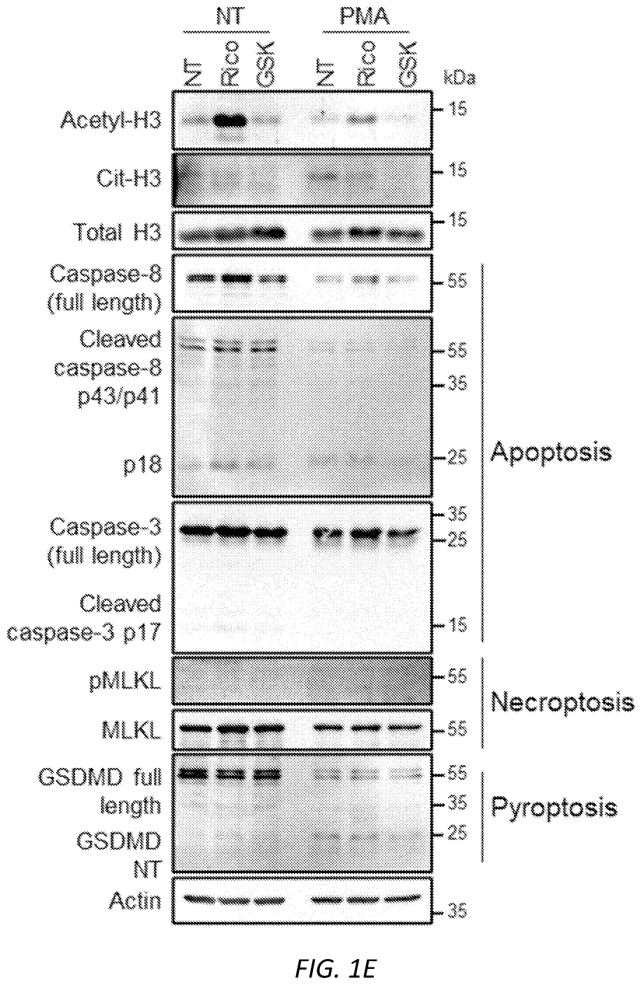

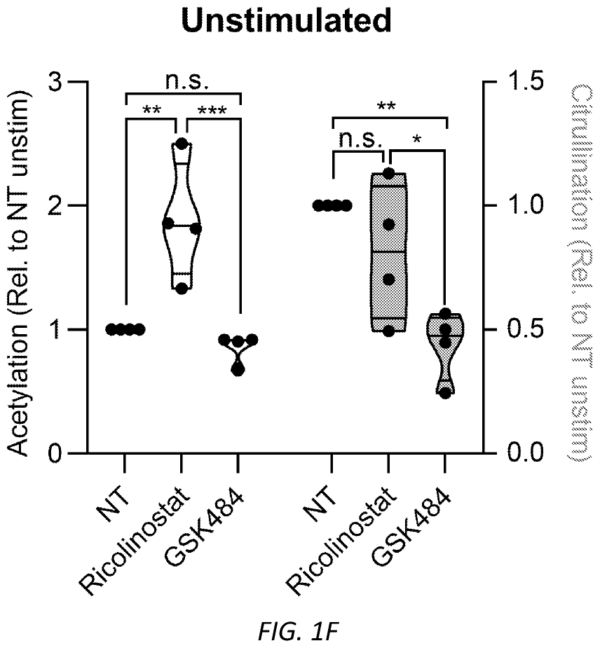

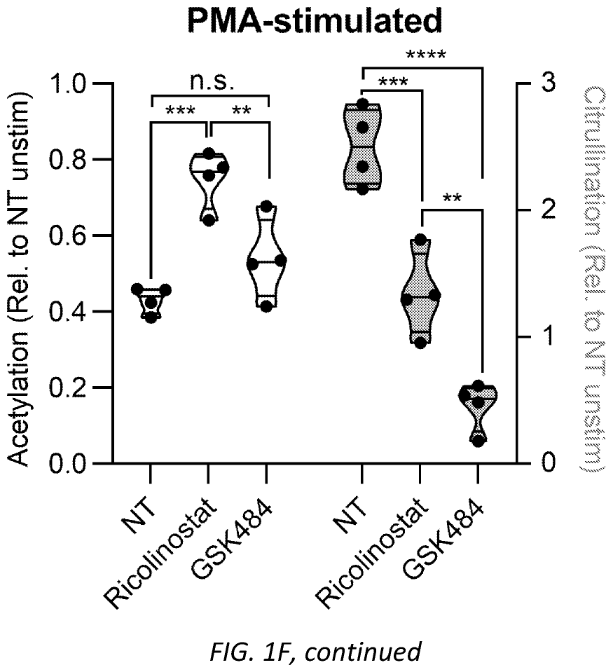

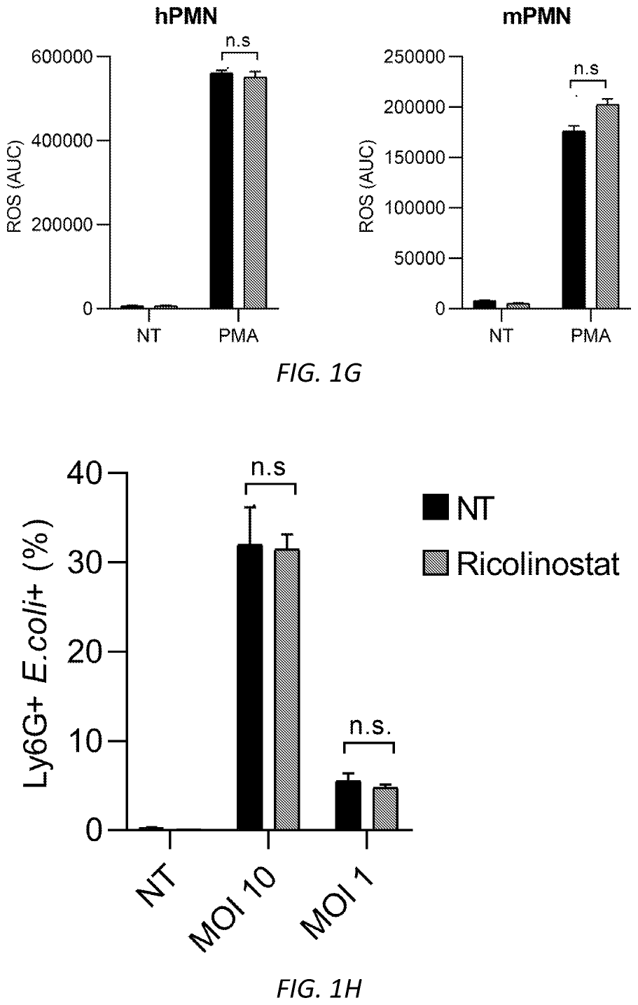

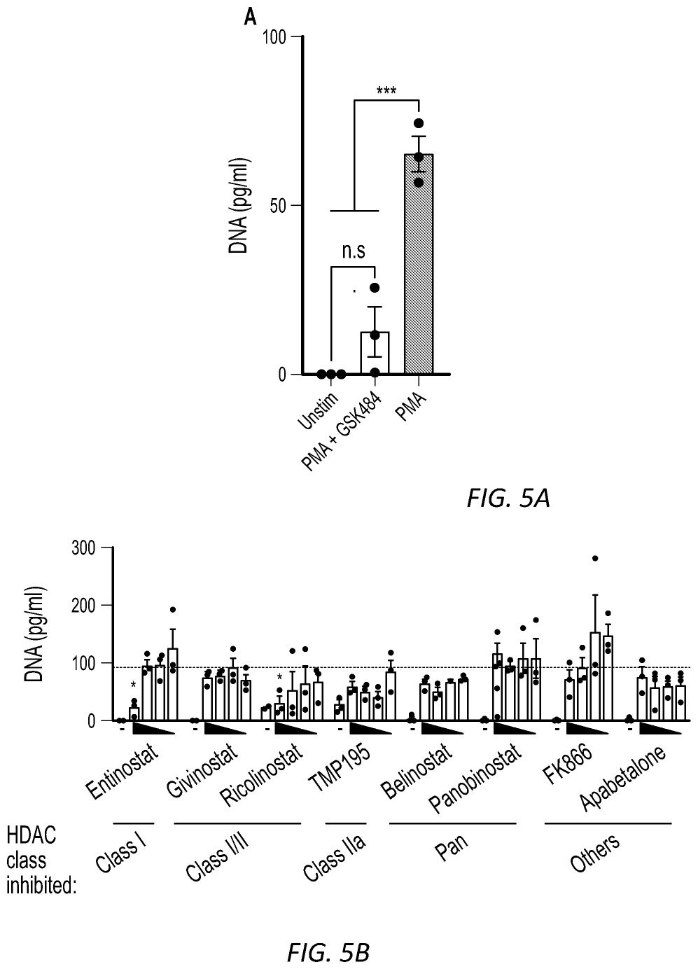

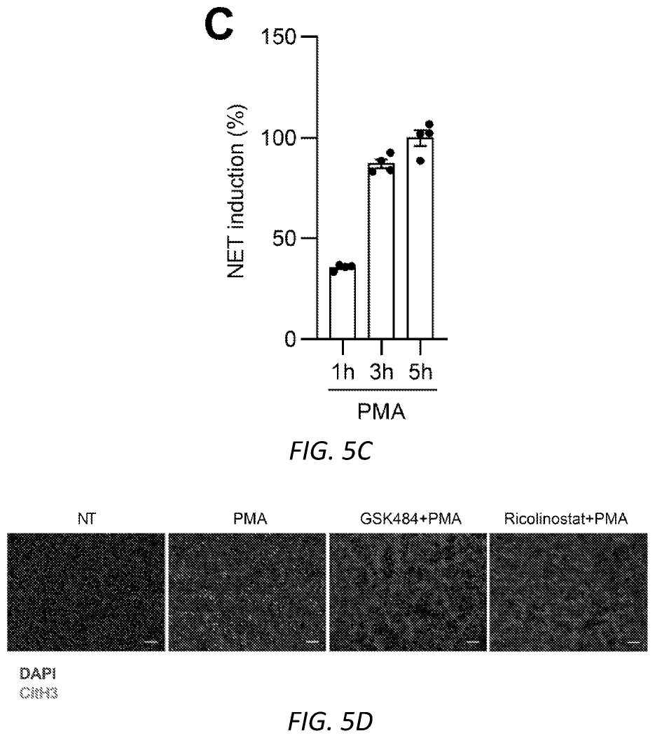

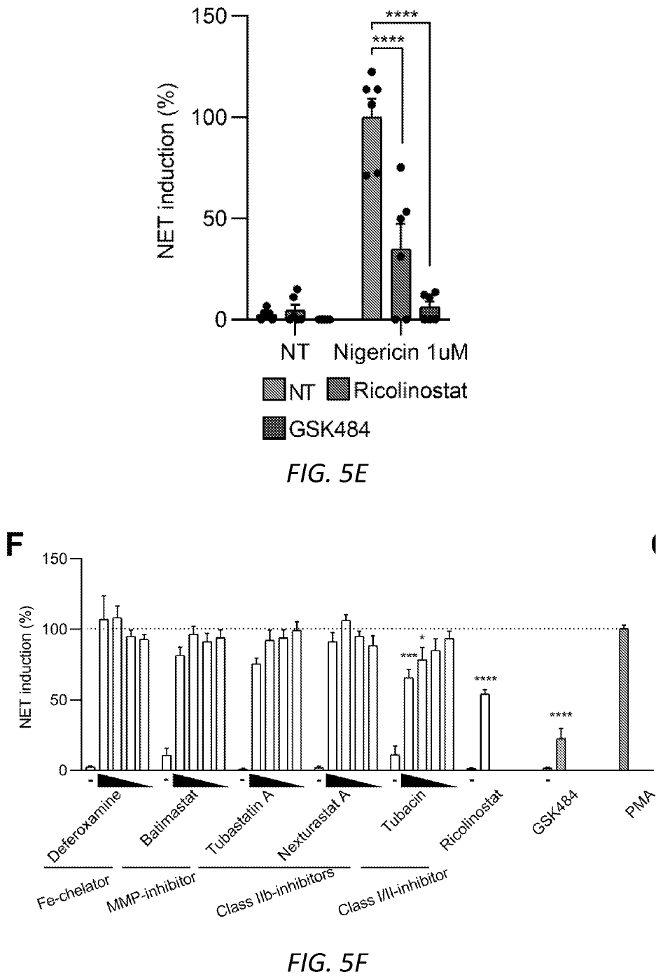

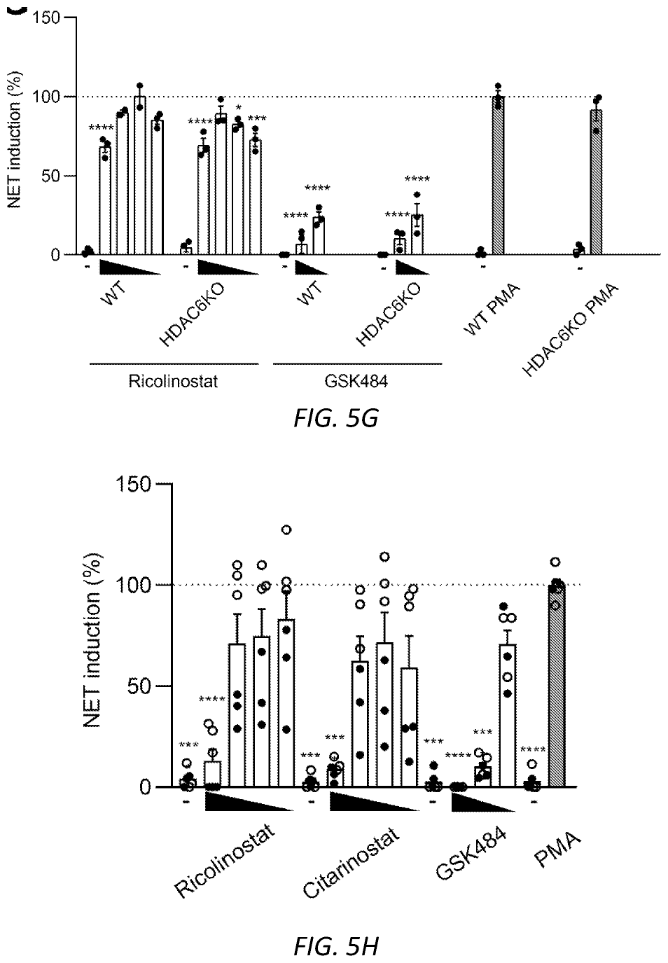

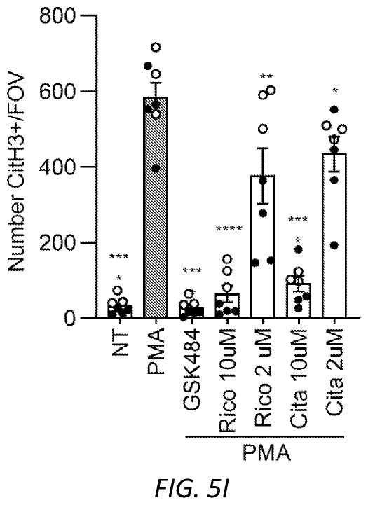

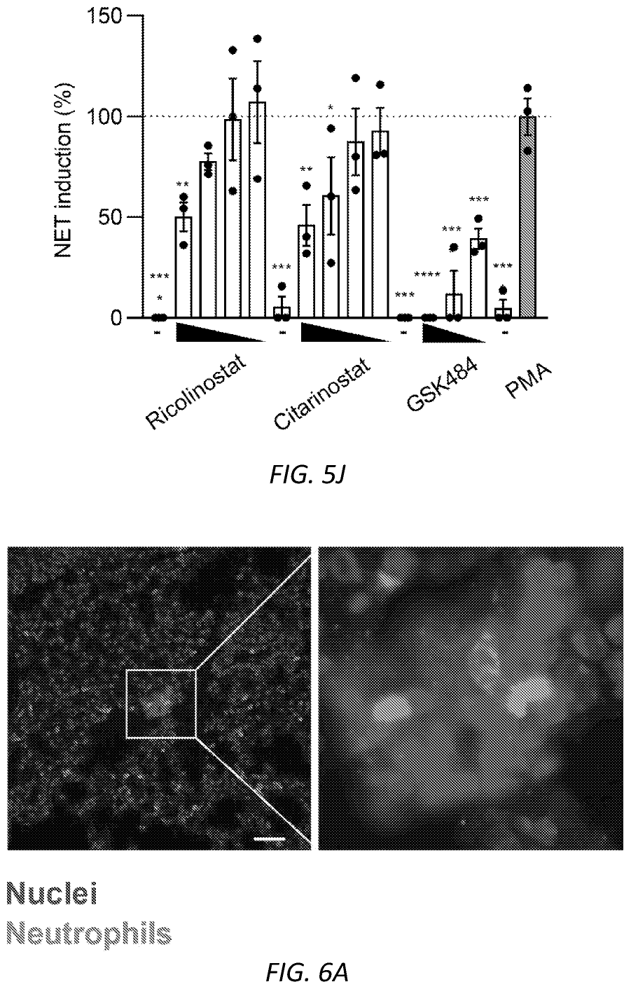

FIGS. 1A-H. Class I/II HDACs drive NET formation
(A and B) Freshly purified human (A) or mouse (B) neutrophils were treated with the indicated HDAC inhibitors at different doses (10, 2, 0.4, 0.08 μM), or with GSK484 (10 μM), for 1 h and then stimulated with 1 μM PMA for 3 h. Cells were fixed and stained with anti-citrullinated histone H3 (CitH3) antibody. NETosis induction was measured as the level of anti-CitH3 fluorescent signal. PMA alone represents 100% of NET induction. “-”: Cells treated with the highest dose of the indicated drug but not activated with PMA.
(C and D) Human neutrophils were treated, or not, with ricolinostat or GSK484 and then stimulated with 1 μM PMA. NETosis induction was measured as area of nuclei stained with DAPI. (C) 3D reconstruction of nuclei in each treatment (grid size 7.8 μm). (D) Quantification of DAPI signal from multiple fields of view (D) (n=9 for unstimulated, not pretreated with any drug [NT] and ricolinostat, n=7 for GSK484).
(E and F) Human neutrophils were treated as in (C-D), and immunoblot analysis of the indicated proteins was performed. Relative quantification of band intensity was analyzed with ImageJ software (F). White violins plots depict the quantification of histone H3 acetylation; red violin plots depict the quantification of histone H3 citrullination. Immunoblot in E is representative of four independent experiments. Gasdermin D NT, gasdermin D N terminal; pMLKL, phospho-MLKL.
(G) Human (left) or murine (right) neutrophils were treated, or not, with ricolinostat (10 μM) and then stimulated with 1 μM PMA for 3 h. ROS production was measured by luminescence with luminol-HRP. Histograms represent the total amount of ROS produced overtime.
(H) Murine neutrophils were treated, or not, with ricolinostat (10 μM) and then stimulated with fluorescent E. coli at the indicated multiplicity of infection (MOI) for 1 h. The histogram represents the percentage of neutrophils positive for E. coli staining. Graphs show mean±SEM. Statistics were calculated using one-way ANOVA (A, B, D) and two-way ANOVA (F, G, H) (*p<0.05; **p<0.01; ****p<0.0001. n.s., not significant). Statistics refers to the comparison with neutrophils stimulated with PMA alone (A, B). Gray stars represent comparison of all experimental groups with the unstimulated control, and black stars represent comparison between drug-treated groups and cells treated with PMA only (NT) (D). See also FIGS. 5A-J.

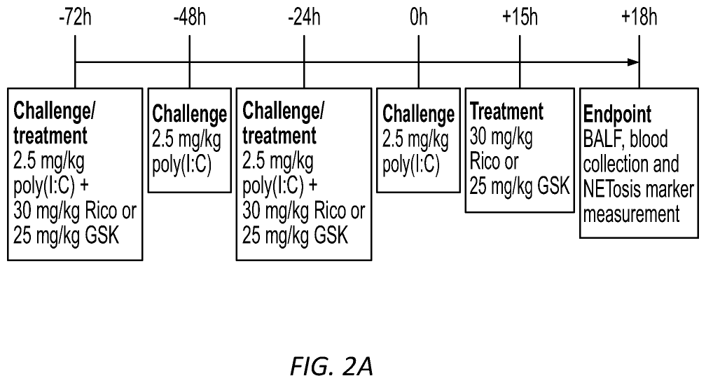

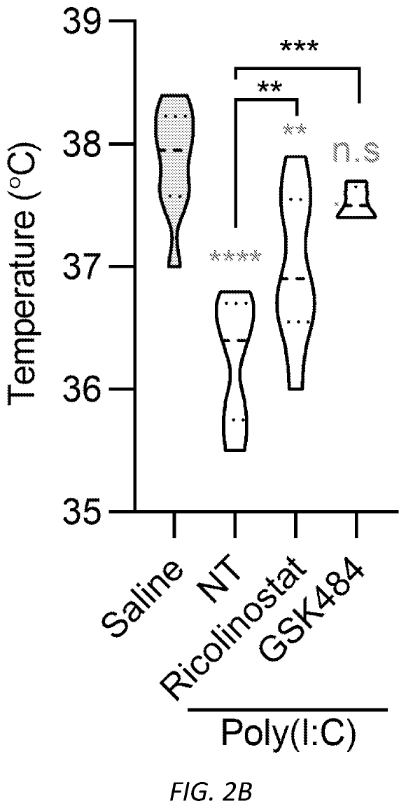

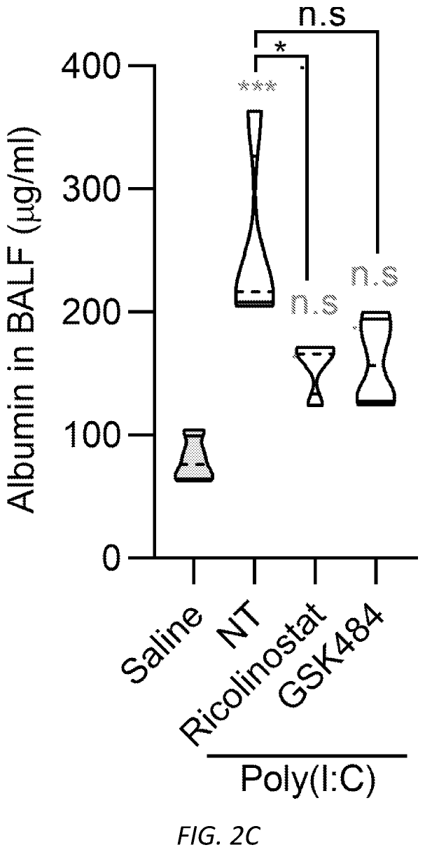

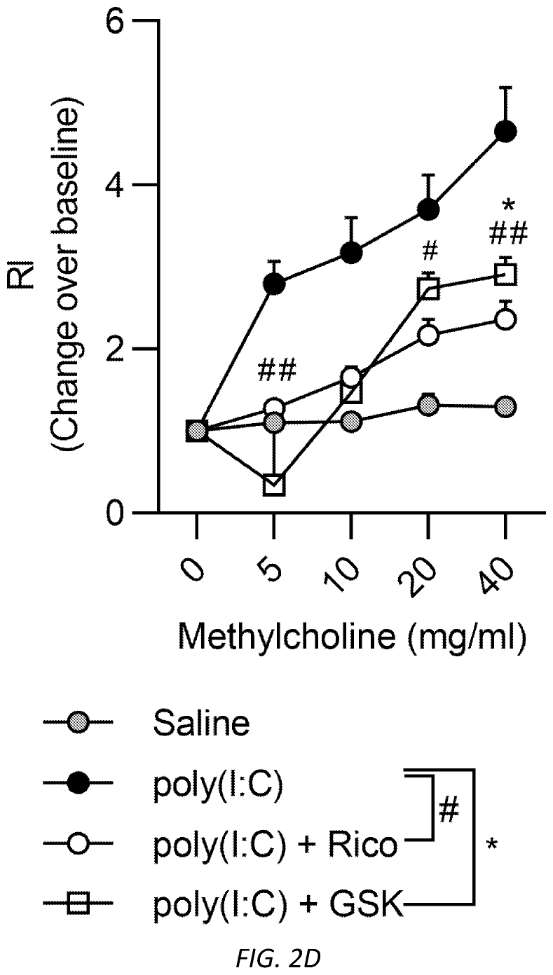

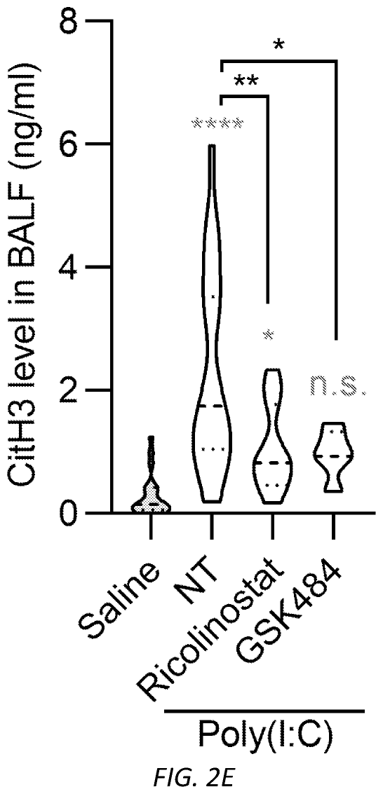

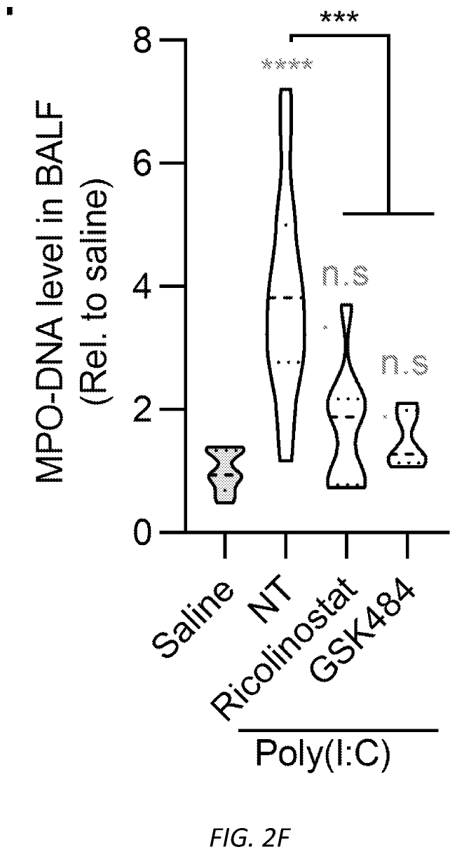

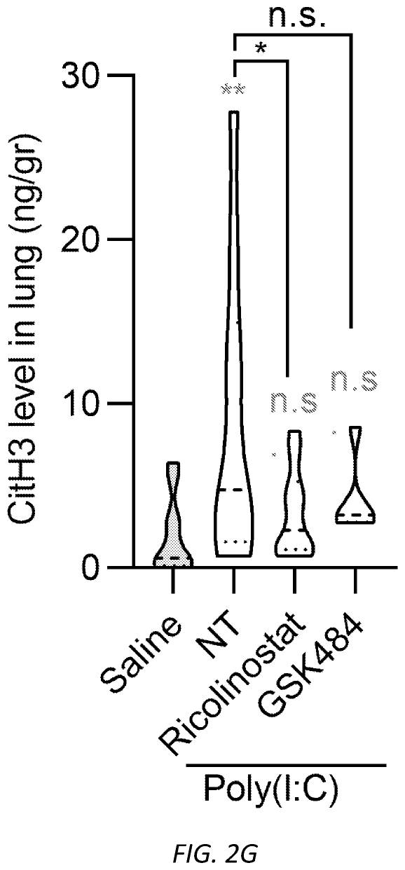

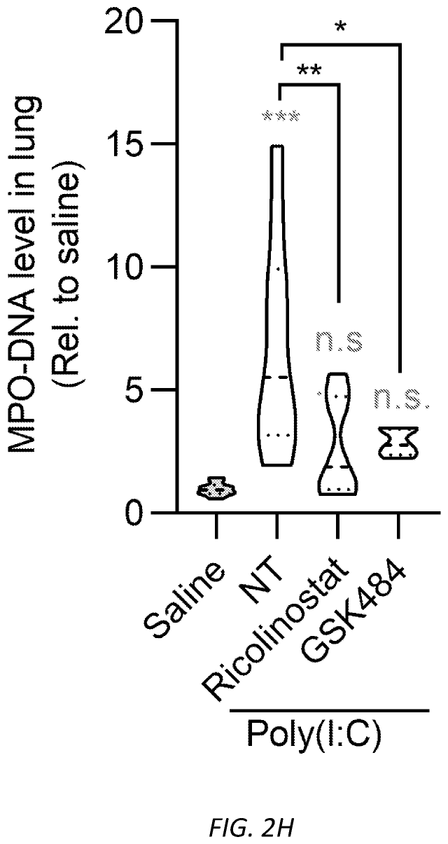

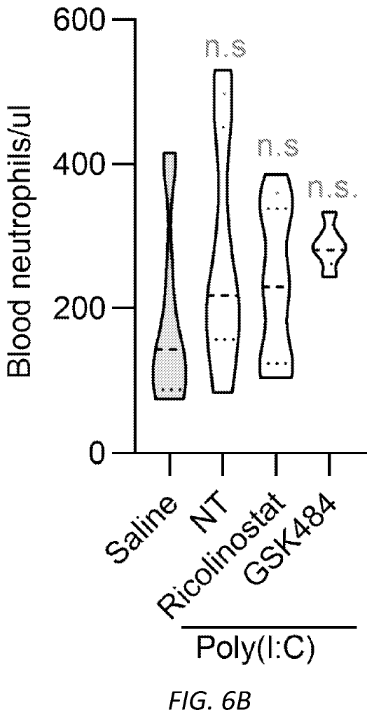

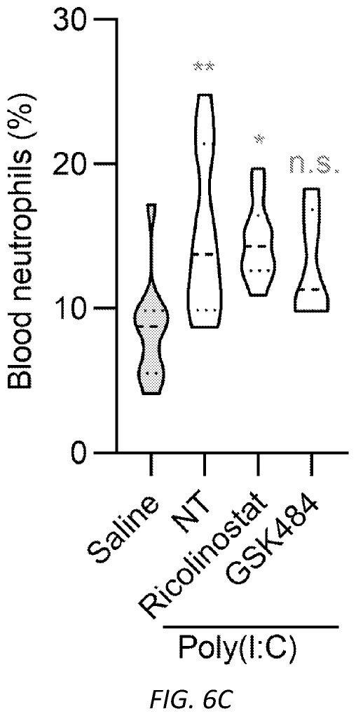

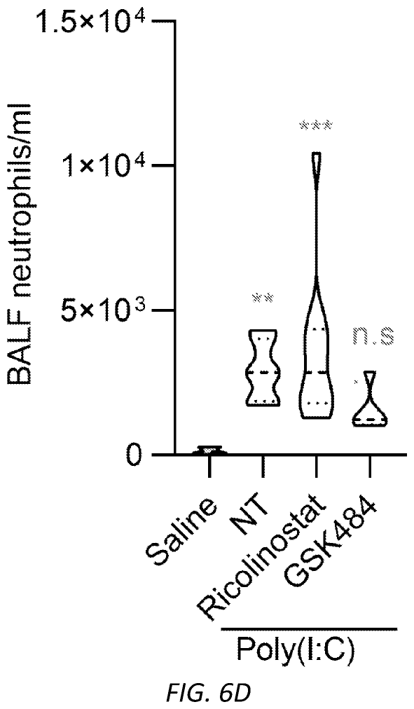

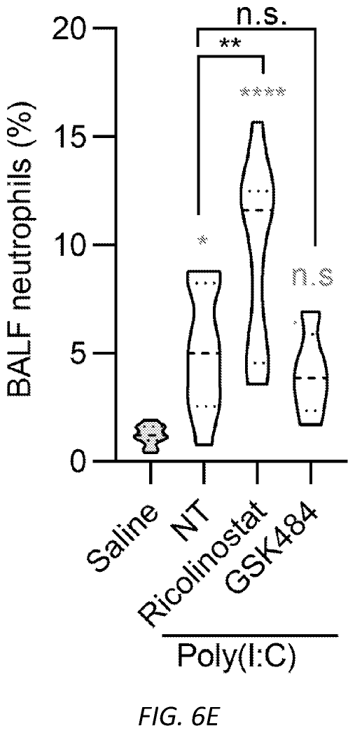

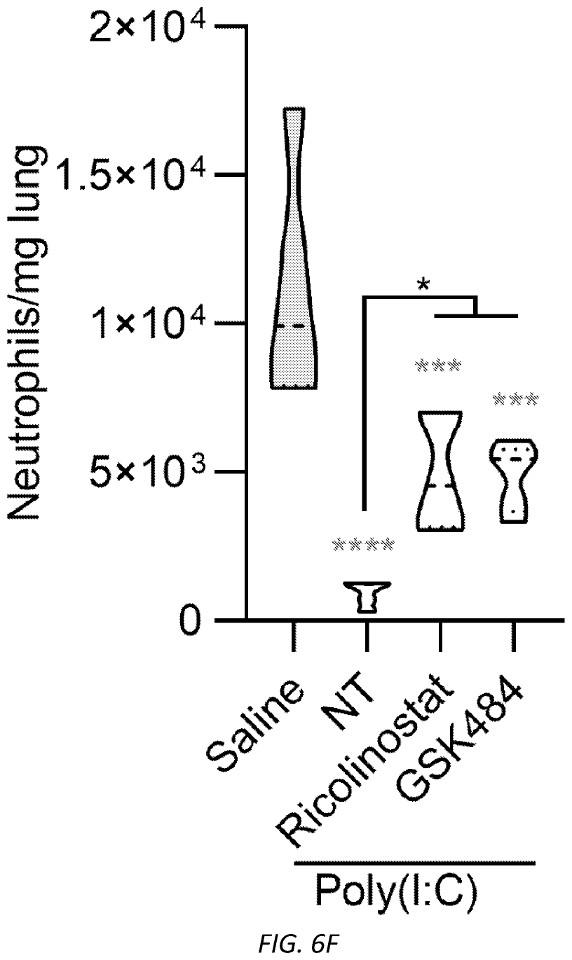

FIGS. 2A-H. Inhibition of class I/IIb HDACs protects against NETosis and ARDS in a mouse model of RNA viral lung infection
(A) Timeline of poly(I:C) instillation and drug treatments. Mice were intratracheally treated with 2.5 mg/kg poly(I:C) or saline daily for 4 days and euthanized 18 h post the last poly(I:C) instillation. Ricolinostat (30 mg/kg/dose) or GSK484 (25/mg/kg/dose) was administered intraperitoneally at day 2, day 4, and 3 h before the endpoint.
(B) Body temperature measurements at the endpoint.
(C) Lung permeability was measured as albumin content level in the BALF (n=5).
(D) Measure of airway hyperreactivity (AHR) in the indicated groups in response to increasing concentration of methacholine. RI, responsiveness index.
(E-H) CitH3 and MPO-DNA levels were analyzed by ELISA in BALFs (E, F) and in lungs (G, H) of mice treated as indicated. The saline group represents mice that received saline instead of poly(I:C). NT group represents mice that were challenged with poly(I:C) without receiving any drug. Statistics were calculated using one-way ANOVA (B, C, E-H) and two-way ANOVA with Tukey's analysis (D) (*p<0.05; **p<0.01; ***p<0.001; ****p<0.0001. n.s., not significant). n=10 (B, D) and 15 (E-H) for each group except for the GSK484-treated group (n=5). Gray stars represent comparison of all experimental groups with the saline-treated control. Black stars represent comparison between drug-treated groups and mice treated with poly(I:C) alone. Violin plots represent median (dashed line) with quartiles (dotted line). See also FIGS. 6A-F.

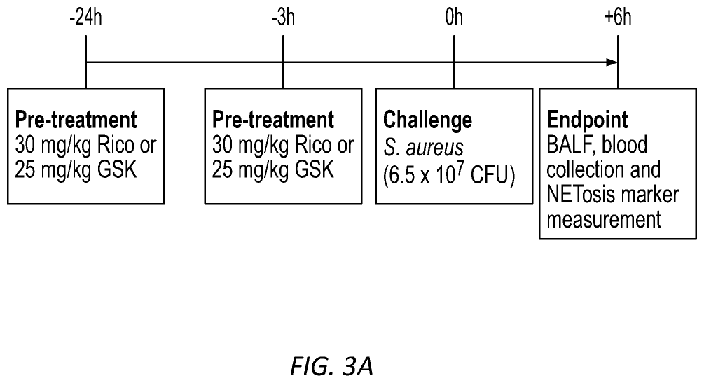

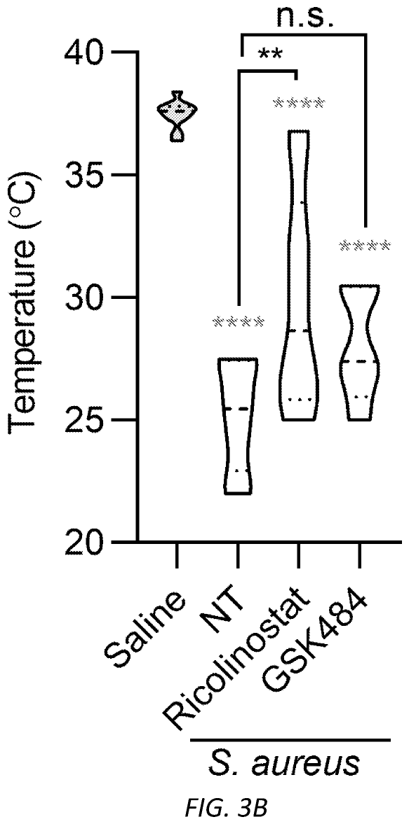

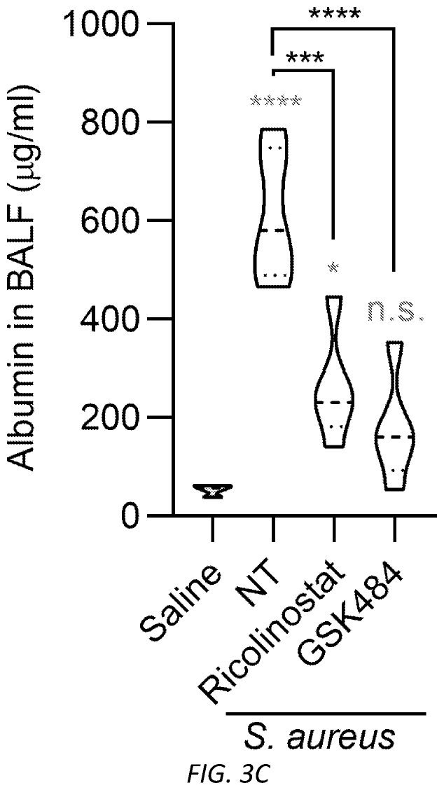

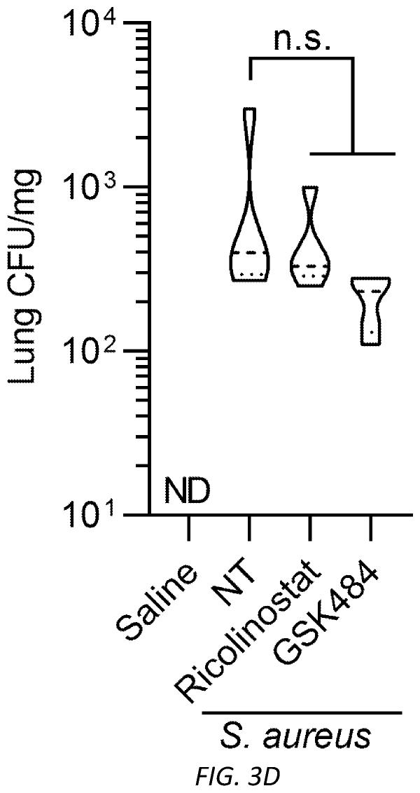

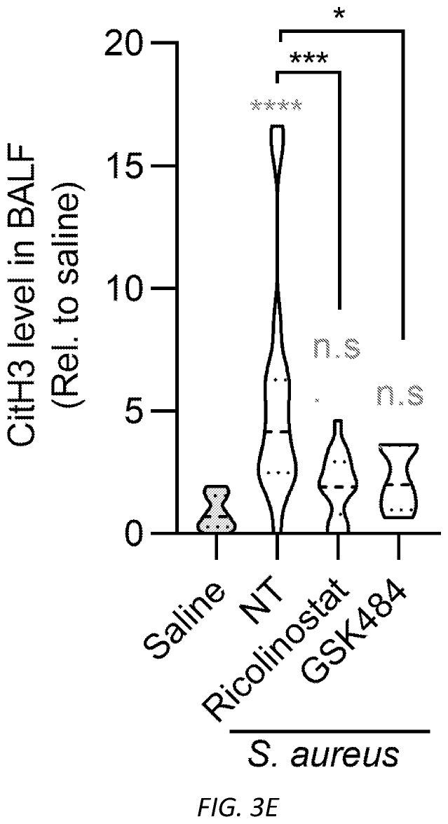

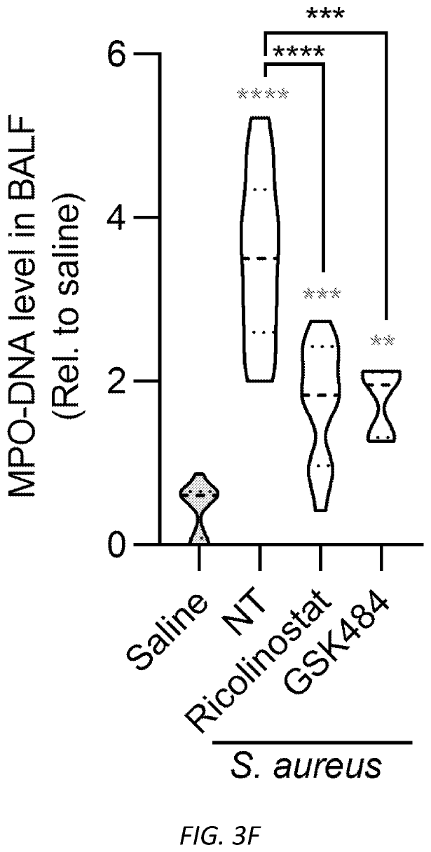

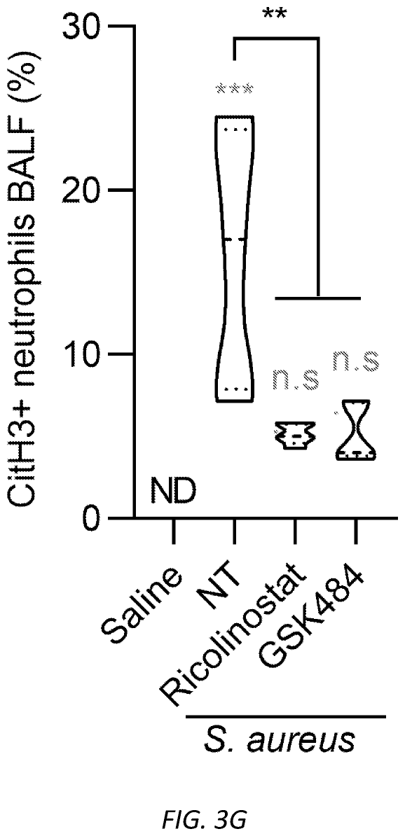

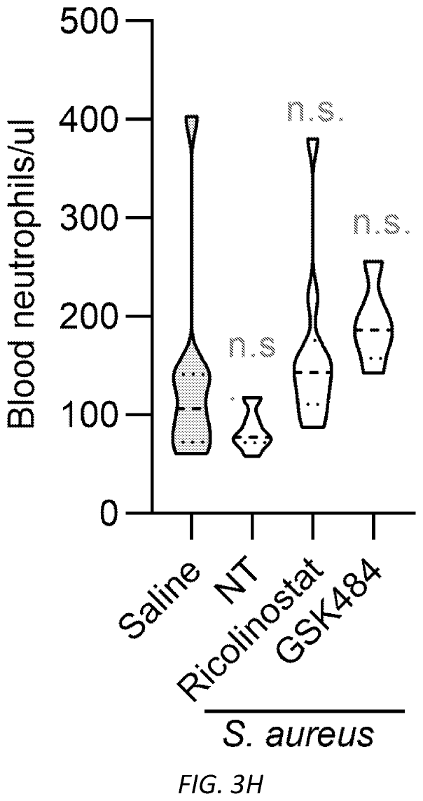

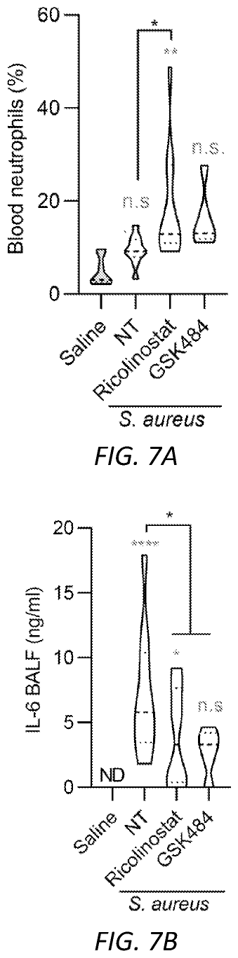

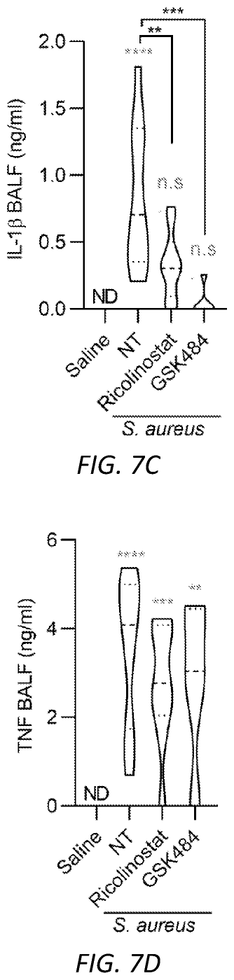

FIGS. 3A-I. Class I/IIb HDACs increase NETosis and ARDS in a mouse model of bacterial pneumonia
(A) Timeline of drug treatment and S. aureus infection. Mice were intraperitoneally injected with two doses of ricolinostat or GSK484 and were intratracheally treated with S. aureus (6.5×10⁷ CFU). Six hours later, BALFs, lungs, and blood were collected and analyzed.
(B) Body temperature measurements at the endpoint.
(C) Lung permeability was measured as albumin content in the BALF (n=5).
(D) Numbers of S. aureus CFU were measured in lung homogenates (n=5).
(E and F) CitH3 and MPO-DNA levels in BALFs were detected by ELISA in the indicated groups and normalized to saline controls.
(G) NETosis levels in BALFs were quantified by cytofluorimetry as neutrophils that were positive for the CitH3 staining (n=5).
(H and I) Neutrophil numbers were analyzed in blood (H) and BALF (I) by cytofluorimetry. The saline group represents healthy mice. The NT group represents mice that were challenged with S. aureus and did not receive any drug. Statistics were calculated using one-way ANOVA (*p<0.05; **p<0.01; ***p<0.01; ****p<0.0001. n.s., not significant). n=10 (B, H, I) and 15 (E, F) for each group except for the GSK484-treated group (n=5). Gray stars represent comparison of all experimental groups with the saline-treated healthy control. Black stars represent comparison between drug-treated groups and mice treated with S. aureus alone. Violin plots represent median (dashed line) with quartiles (dotted line). See also FIGS. 7A-D.

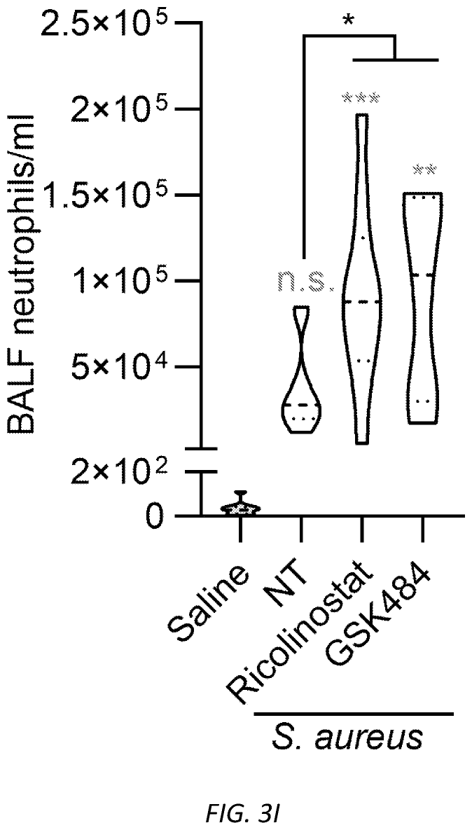

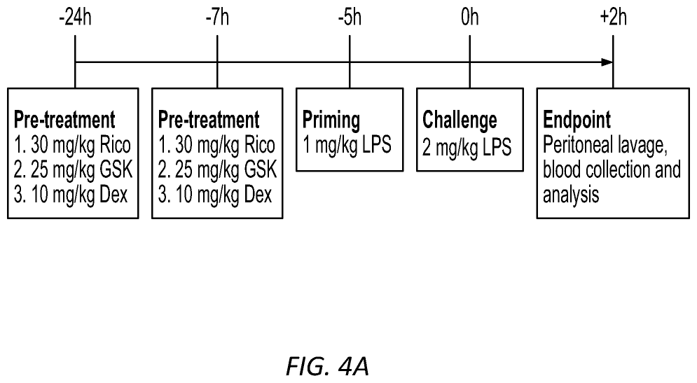

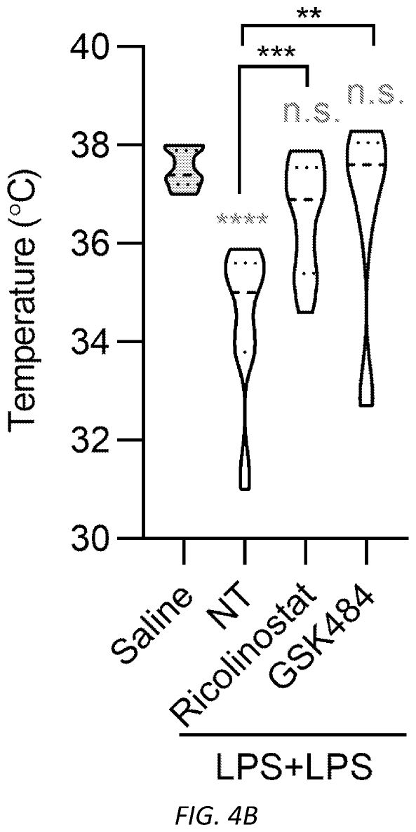

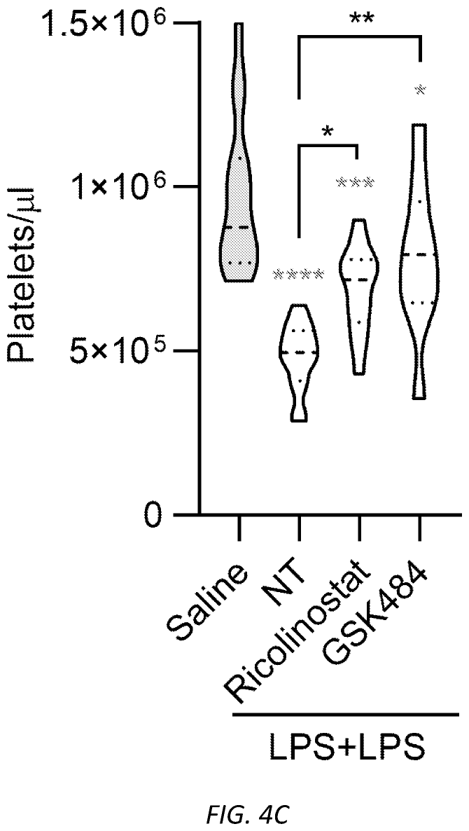

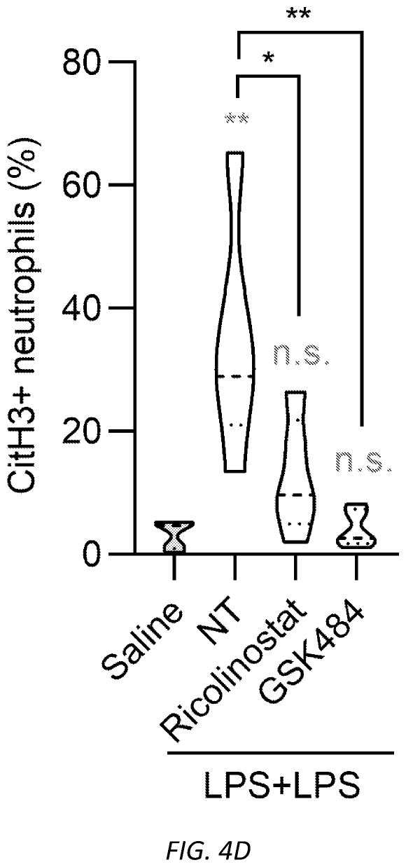

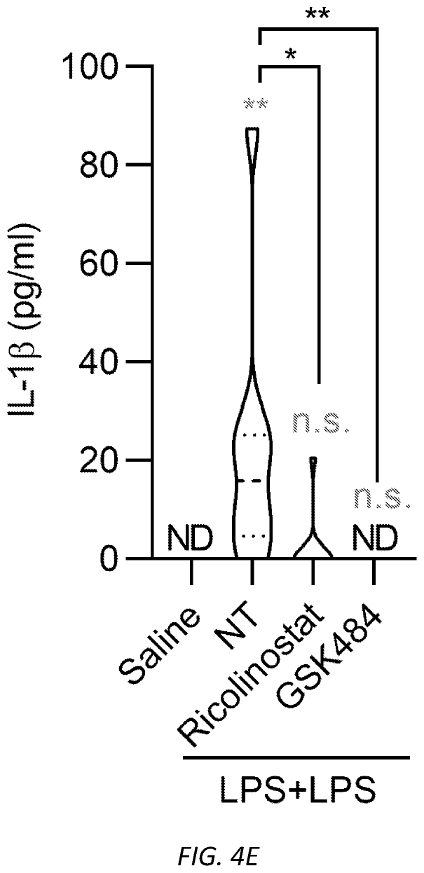

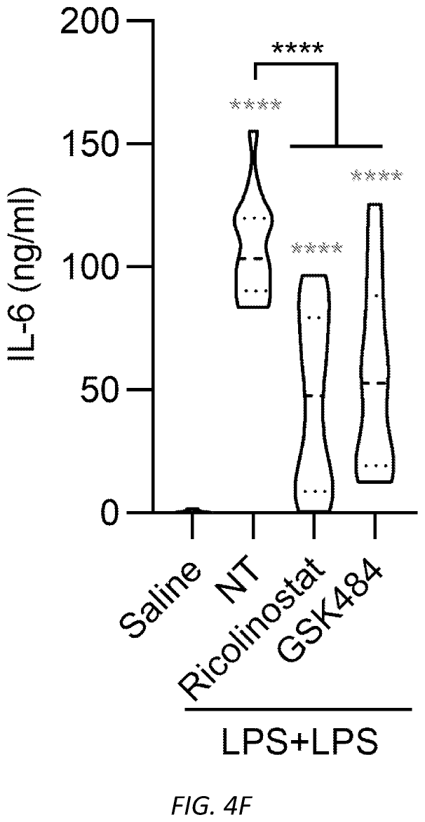

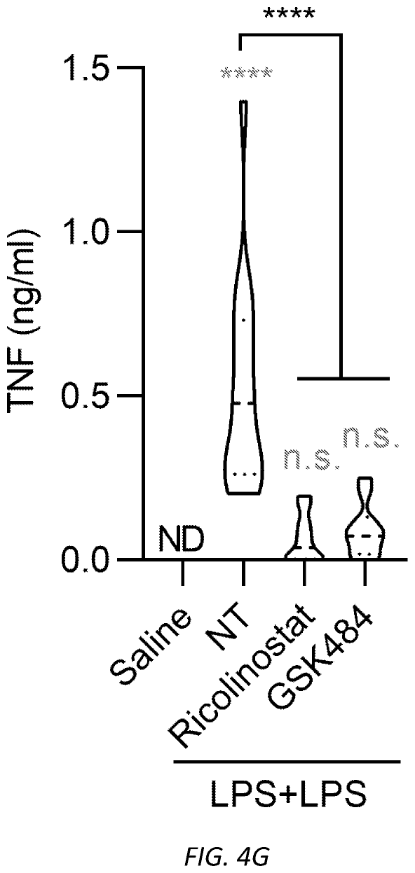

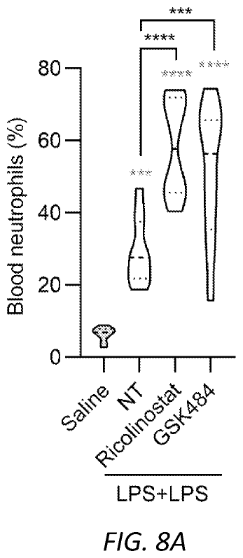

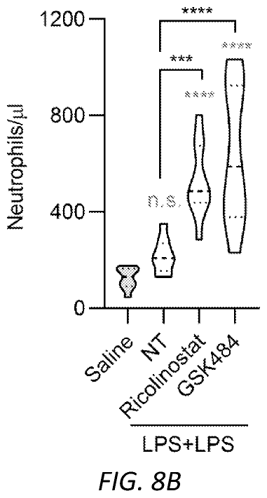

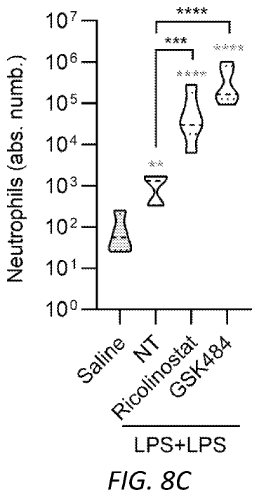

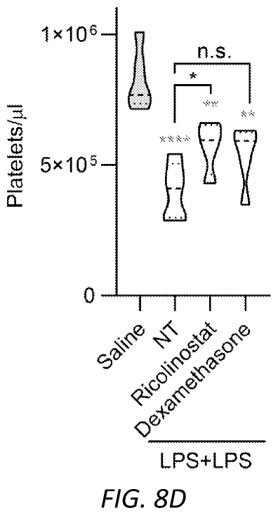

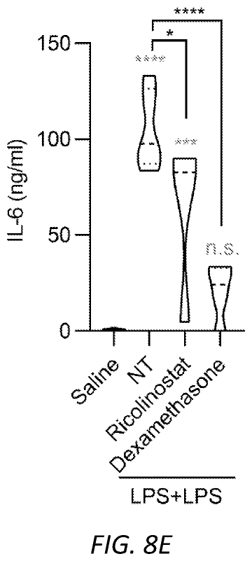

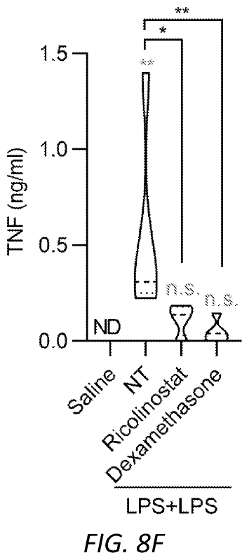

FIGS. 4A-G. Class I/IIb HDAC inhibition protects against systemic inflammation
(A) Time line of drug treatment and LPS priming and challenge. Mice were intraperitoneally injected, or not, with two doses of ricolinostat or GSK484 and were then intraperitoneally administered with two doses of LPS (−5 h, 1 mg/kg and 0 h, 2 mg/kg) to induce lethal sepsis. Peritoneal lavage and blood were collected 2 h after the LPS challenge.
(B) Body temperature was measured 2 h post challenge.
(C) Concentration of platelets in the blood was measured by cytofluorimetry.
(D) NETosis was measured as neutrophils (Ly6G+/CD11b+) positive for citrullinated histone H3 staining in the peritoneal lavage.
(E-G) Serum cytokines were measured by ELISA. The NT group represents mice that were challenged with LPS and did not receive any drug. Statistics were calculated using one-way ANOVA (*p<0.05; ** p<0.01; *** p<0.001; ****p<0.0001; n.s., not significant). n=15 for each group except the GSK484-treated group (n=10). Gray stars represent comparison of all experimental groups with the saline-treated healthy control. Black stars represent comparison of drug-treated groups with LPS-stimulated mice without drug treatment. Violin plots represent median (dashed line) with quartiles (dotted line). See also FIGS. 8A-F.

FIGS. 5A-J. Class I/II HDACs drive NET formation.
A) Quantification of extracellular DNA released by neutrophils treated, or not, with 1 μM GSK484 for 1 h and then stimulated with 1 μM PMA for 4 hours. Unstimulated, neutrophils not treated with drugs were used as a control.
B) Freshly purified human neutrophils were treated with the indicated HDAC inhibitors at different doses (0.08, 0.4, 2 and 10 μM) for 1 hour. Extracellular DNA release was assessed in the supernatant 3 hours after PMA stimulation. The dotted line represents the amount of DNA released from neutrophils treated with PMA alone. “-”: cells treated with the highest dose of the indicated drug but not activated with PMA.
C) Freshly purified human neutrophils were stimulated for 1 h, 3 h or 5 h. Cells were fixed and stained with an anti-CitH3 antibody. NETosis induction was measured as level of anti-CitH3 fluorescent signal. 5h-PMA treatment represents 100% NET induction.
D) Representative images of freshly isolated human neutrophils treated or not with 10 μM of the indicated drugs and stimulated with 1 μM PMA for 3 hours. Cells were fixed and then stained for CitH3 (red) and nuclei (blue). Scale bar=100 μm.
E) Murine neutrophils were treated with ricolinostat or GSK484 and then stimulated with 1 M nigericin for 3 hours. NET induction was measured as in (C). Nigericin alone represents 100% NET induction.
F) Freshly purified human neutrophils were treated with the indicated drugs at different doses (10, 2, 0.4, 0.08 μM), ricolinostat or with GSK484, for 1 hour and then stimulated with 1 μM PMA for 3 hours. Cells were fixed and stained with an anti-CitH3 antibody. NETosis induction was measured as level of anti-CitH3 fluorescent signal. PMA alone represents 100% of NET induction. “-”: cells treated with the highest dose of the indicated drug but not activated with PMA.
G) Neutrophils purified from wild-type or HDAC6KO mice were treated with different concentration of ricolinostat (10, 2, 0.4, 0.08 μM) or GSK484 (10, 2 μM) and then stimulated with PMA. NET induction was measured as in (F).
H, I) Freshly isolated neutrophils from two different donors were treated with different doses of ricolinostat, citarinostat (10, 2, 0.4, 0.08 μM) or GSK484 (10, 5, 2 μM) for 1 h and then stimulated with 1 μM PMA. NET induction was measured as level of anti-CitH3 fluorescent signal (H) and by microscopy as number of cells positive for CitH3 signal per field of view (FOV) (I). Black dots represent donor 1, white dots donor 2. n=7 images representing different areas within the well (I).
J) Murine neutrophils were treated as in G). NET induction was measured as level of anti-CitH3 fluorescent signal. PMA alone represents 100% of NET induction. “-”: cells treated with the highest dose of the indicated drug but not activated with PMA. Graph shows mean±SEM. Statistics were calculated using One-way ANOVA (A, E, I) and Two-way ANOVA (B,, F, G, H, L). (*P<0.05; **P<0.01; ***P<0.001 ****P<0.0001. n.s., not significant). Statistic refers to the comparison with neutrophils stimulated with PMA alone.

FIGS. 6A-F. Inhibition of class I/IIb HDACs protects against NETosis and ARDS in a mouse model of RNA viral lung infection.
A) Representative image of lung section of mice challenged with poly(I:C). Nuclei are stained with DAPI (blue), and anti-neutrophils antibody (red). Scale bar=100 μm.
B-F) Neutrophils numbers and percentage were calculated in blood (B, C), BALFs (D, E), and lung tissues (F) of mice challenged, or not, with poly(I:C). The saline group represents mice that received saline instead of poly (I:C). NT group represent mice that were challenged with poly (I:C) without receiving any drug. Ricolinostat (30 mg/kg/dose) or GSK484 (25 mg/kg/dose) were administered intraperitoneally at day 2, day 4 and 3 h prior the endpoint. Violin plots represent median (dashed line) with quartiles (dotted line). Statistics were calculated using One-way ANOVA (*P<0.05; **P<0.01; ***P<0.001; ****P<0.0001; n.s., not significant). Grey stars represent comparison of all experimental groups with the saline control. Black stars represent comparison of drug treated groups with NT group.

FIGS. 7A-D. Class I/IIb HDACs increase NETosis and ARDS in a mouse model of bacterial pneumonia.
A) Neutrophil percentage in mice challenged, or not, with S. aureus was determined by cytofluorimetry. Saline group represent healthy mice. NT group represents mice that were challenged with S. aureus and did not received any drug. Ricolinostat (30 mg/kg/dose) or GSK484 (25 mg/kg/dose) were administered intraperitoneally at-24 hours and-3 hours prior challenge.
B-D) Absolute amounts of serum cytokines were measured by ELISA. Violin plots represent median (dashed line) with quartiles (dotted line). Statistics were calculated using One-way ANOVA (*P<0.05; **P<0.01; ***P<0.001; ****P<0.0001; n.s., not significant). Grey stars represent comparison of all experimental groups with the saline control. Black stars represent comparison of drug treated groups with NT group.

FIGS. 8A-F. Class I/IIb HDAC inhibition protects against systemic inflammation.
A-C) Neutrophils numbers and percentage were calculated in blood (A, B) and peritoneal lavage (C) of mice challenged, or not, with LPS. Saline group represent healthy mice. NT group represent mice that were challenged with LPS without receiving any drug. Ricolinostat (30 mg/kg/dose) or GSK484 (25 mg/kg/dose) were administered intraperitoneally at day −1, and 2 hour prior LPS priming.
D) Concentration of platelets in the blood was assessed by cytofluorimetry 2 hours post LPS-challenge.
E, F) Absolute amounts of serum cytokines were measured by ELISA. Violin plots represent median (dashed line) with quartiles (dotted line). Statistics were calculated using One-way ANOVA (*P<0.05; **P<0.01; ***P<0.001; ****P<0.0001; n.s., not significant). Grey stars represent comparison of all experimental groups with the saline control. Black stars represent comparison of drug treated groups with NT group.

### DETAILED DESCRIPTION

Akin to transcriptional activation that is driven by local chromatin remodeling, epigenetic modifications of histones that decondense chromatin are a prerequisite for NET release (Kenny et al., 2017). In particular, histone citrullination by the peptidylarginine deiminase 4 (PAD4) has been shown to facilitate NET formation and release (Thiam et al., 2020; Wang et al., 2009). Granule-resident serine proteases, neutrophil elastase, as well as gasdermin D also participate to chromatin decondensation, nuclear expansion, and formation of NETs (Chen et al., 2018; Papayannopoulos et al., 2010; Sollberger et al., 2018). Whether other epigenetic modifications also participate in NETosis, and how they may be linked to the above-mentioned processes that alter the structure of chromatin, remains largely overlooked. Based on the importance of histone acetylation in determining chromatin structure and accessibility, we hypothesized that enzymes involved in histone acetylation also contribute to NETosis. Histone acetylation is dynamically controlled by two counteracting protein families, the histone acetyltransferases and the histone deacetylases (HDACs) as “writers” and “erasers” (Grunstein, 1997; Zhao et al., 2018). HDACs belong to two families, based on the dependency on zinc or nicotinamide adenine dinucleotide (NAD+) for their activities (Seto and Yoshida, 2014).

When infection and tissue damage coincide, the immune system potently reacts to avoid the spread of the infection, even at the cost of further damage. Neutrophils are central to this process, thanks to the release of NETs that contain and restricts viral, bacterial, as well as fungal infections (Brinkmann et al., 2004; Saitoh et al., 2012; Thiam et al., 2020). Nevertheless, excessive tissue damage caused by NET release has been often associated with immune-driven disorders (Thiam et al., 2020). Moreover, NETosis has been shown to play both protective as well as detrimental roles during tissue repair and resolution (Hahn et al., 2019; Kang et al., 2020; Schauer et al., 2014). Thus, a better understanding of the mechanisms that regulate NET induction is fundamental to develop future possible therapeutic ways of intervention to either potentiate or block the induction of NETs.

Data herein show that class I/IIb HDACs drive the release of NETs by human and mouse neutrophils. Their inhibition in mice dampens inflammation and protects against microbial pneumonia or septic shock. These data establish a new biological feature of a group of HDACs whose activity is well known to regulate gene transcription and open to the use of drugs that inhibit HDACs to restrain the tissue-damaging functions of neutrophils. Although targeting of PAD4 with the chemical compound GSK484 was previously shown to be effective in inhibiting CitH3 and NET formation, the present results demonstrate that ricolinostat, a drug currently utilized in a phase II clinical trial, can also be used to efficiently inhibit NETosis and inflammation. In multiple in vivo mouse models mimicking bacterial- or viral-induced infections and septic shock, pharmacological inhibition of HDACs ameliorated impaired lung functionality, reduced systemic inflammation, and decreased thrombosis; without wishing to be bound by theory, it appears that these effects occur via inhibition of NETosis.

Viral or bacterial-induced pneumonia and COVID-19 have a strong correlation to elevated levels of circulating neutrophils, NETs, and inflammatory cytokines that hint at a chronic inflammatory storm in patients (Mehta et al., 2020). NET formation is intimately linked to thrombosis that drives microvasculature damages in lung and other organs (Ackermann et al., 2020; Al-Samkari et al., 2020; Tang et al., 2020). Neutrophils and NETosis also participate in the most severe cases of COVID-19 (Zuo et al., 2020a, Zuo et al., 2020b). NETs infiltrate lungs of patients with severe COVID-19, and SARS-COV-2 has been shown to trigger the release of NETs that mediate lung pathology (Middleton et al., 2020; Radermecker et al., 2020; Veras et al., 2020). As dysregulated production of NETs is known to exacerbate inflammation and accelerate the pathogenesis of ARDS and sepsis caused by other coronaviruses (Blondonnet et al., 2016; Perlman and Dandekar, 2005), these findings support the hypothesis that targeting NET formation represents a viable strategy to alleviate tissue damage and hyperinflammation. Therefore, the present results imply that pharmacological inhibition of HDAC activity may be beneficial for patients with COVID-19 by blocking NETosis.

In mouse models, drugs that inhibit class I/IIb HDACs partially reduced the levels of pro-inflammatory cytokines including IL-1β, TNF, and IL-6. Overall, the present data demonstrate that zinc-dependent lysine deacetylase inhibition prevents NET formation by neutrophils, which in turn reduces systemic inflammation. Thus, provided herein are methods in which HDAC inhibition is used as a therapeutic strategy for the treatment of a variety of diseases mediated by the overproduction of NETs such as COVID-19, pulmonary diseases, autoimmune diseases (e.g., rheumatoid arthritis and systemic lupus erythematosus), diabetes, and cancers, e.g., tumor-associated inflammation, diabetes, cardiometabolic/cardiovascular diseases, anaphylaxis and pancreatitis (Arpinati et al., 2020; Lee et al., 2017; Porto and Stein, 2016; Wong et al., 2015).

### Methods of Treatment

The methods described herein include methods for the treatment or prophylaxis of subjects, e.g., normal subjects, or subjects who have autoimmune diseases (e.g., Antineutrophil cytoplasmic antibody (ANCA)-associated vasculitis (AAV), Type 1 diabetes mellitus (T1DM), Systemic lupus erythematosus (SLE), Rheumatoid Arthritis (RA), psoriasis, Antiphospholipid syndrome (APS), multiple sclerosis (MS), dermatomyositis (DM), polymyositis (PM), and IgG4-related autoimmune pancreatitis (AIP), or Drug-Induced Autoimmune Diseases, see He et al,. Chin Med J (Engl). 2018 Jul. 5; 131(13):1513-1519; Lee et al., Autoimmun Rev. 2017 November; 16(11):1160-1173)); a cardiovascular event, e.g., thrombosis or myocardial infarction (MI), or in cardiovascular conditions such as small vessel vasculitis (SVV) or atherosclerosis; inflammatory conditions such as gout or Inflammatory bowel diseases (IBDs); complications of infection, e.g., viral or bacterial infection, e.g., viral or bacterial-induced pneumonia and consequences of COVID-19 infection, e.g., associated with cytokine storms; or cancer (see, e.g., Olsson and Cedarvall, Front Immunol. 2016; 7:373; Cedarvall and Olsson, Oncoscience. 2015; 2(11):900-901). The methods can be used, e.g., for reducing NETosis, or reducing the risk of NETosis-related conditions. Generally, the methods include administering a therapeutically effective amount of an inhibitor of class I/IIb HDACs as described herein, to a subject who is in need of, or who has been determined to be in need of (e.g., has been diagnosed with or determined to be at increased risk of developing a condition described herein), such treatment. In some embodiments, the subject does not have cancer, e.g., does not have a solid tumor (such as hepatocellular carcinoma) or a hematopoietic cancer, e.g., leukemia or lymphoma, e.g., multiple myeloma, acute myeloid leukemia, or B-cell lymphoma. In some embodiments, the subject does not have diabetic peripheral neuropathy.

Methods for identifying subjects who have a condition described herein are known in the art. Based on information known in the art a skilled healthcare practitioner could readily make a diagnosis. See, e.g., McMullin et al., Br J Haematol. 2018 Nov. 27. doi: 10.1111/bjh. 15648; Steensma et al., Blood. 2015 Jul. 2; 126(1):9-16; Heuser et al., Dtsch Arztebl Int. 2016 May 6; 113(18):317-22 Tefferi and Barbui, Am J Hematol. 2015 February; 90(2):162-73; Double and Harrison, Hematology. 2015 Mar; 20(2):119-20. In some embodiments, the subject has a perforin mutation (e.g., has been identified as having a perforin mutation) or has serum ferritin levels above a threshold.

In some embodiments, the methods can include detecting the presence of NETosis in a subject using methods known in the art (e.g., circulating cfDNA,

MPO/cfDNA conjugates and/or citH3 in serum, see Mutua and Gershwin, Clin Rev Allergy Immunol. 2021; 61(2):194-211), selecting a subject who has NETosis (e.g., based on the presence of a level of cfDNA, MPO/cfDNA conjugates and/or citH3 above a reference level, e.g., a reference level that represents a level of cfDNA, MPO/cfDNA conjugates and/or citH3 in a subject who does not have NETosis), and treating the subject using a method as described herein.

In some embodiments, the methods can include detecting the presence of a mutation in an allele of perforin in the subject, detecting serum ferritin levels and identifying a subject who has a serum ferritin level above a threshold (e.g., a threshold level that represents a level in subjects who do not have or are not at risk of developing cytokine storm syndrome, such that a level above the threshold indicates that the subject has or is at risk of developing cytokine storm syndrome), or identifying a subject who is prone to infection, e.g., subjects who are on immunosuppressants, or who are at increased risk of poor outcome, e.g., subjects who have immune disorders, are elderly (age 65 or older), or who live in in community housing facility such as a senior care home.

Subjects with perforin mutations are believed to be more susceptible to cytokine storms, which are related to NETosis; see, e.g., Schulert et al., J Infect Dis. 2016 Apr. 1; 213(7):1180-1188, found evidence of perforin pathway-related mutations in five of the 14 samples they analyzed from patients who died in the 2009 H1N1 flu outbreak. Cytokine storm syndrome was identified as a potential contributing factor in this abnormally high number of deaths in otherwise healthy individuals.

In some embodiments, the methods can include detecting the presence of damage-associated molecular patterns (DAMPs) or pro-inflammatory cytokines such as high mobility group box-1 (HMGB1), which plays a critical roles in the pathogenesis of several different inflammatory diseases (Amini et al., Rep Biochem Mol Biol. 2019 January; 7(2):204-209). HMGB1 is a sterile inflammatory molecule released from various cells during stress has been implicated in inflammation (Gonelevue et al., Stress J Vasc Res 2018; 55:244-254. The presence of DAMPs, e.g. HMGB1, above a threshold level can be used to identify subjects for treatment or prophylaxis using a method described herein.

Suitable reference values for the above can be determined using methods known in the art, e.g., using standard clinical trial methodology and statistical analysis. The reference values can have any relevant form. In some cases, the reference comprises a predetermined value for a meaningful level, e.g., a control reference level that represents a normal level of the marker, e.g., a level in an unaffected subject or a subject who is not at risk of developing a disease described herein, and/or a disease reference that represents a level associated with conditions associated with NETosis.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

The present methods can also include administering other treatments, e.g., anti-inflammatory drugs and/or anti-viral drugs.

### Inhibitors of Class I/IIb HDACs

Inhibitors useful in the methods described herein include small molecule inhibitors of class I (HDAC1/2/3/8) and class IIb (HDAC6/10) inhibitors. Suitable inhibitors include entinostat, ricolinostat and its analog citarinostat, and AES-135 (Shouksmith et al., ACS Med Chem Lett. 2020 Jan. 9; 11(1):56-64); HDAC-IN-3 and analogues (WO/2008040934); ACY-738; Abexinostat; CAY10603; WT-161; EDO-S101; UF010; Resminostat; HPOB; or CRA-026440.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to reduce the risk of or delay onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising or consisting of small molecule inhibitors of class I and/or class IIb HDACs as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., other anti-inflammatory drugs and/or anti-viral drugs. For example, provided herein are compositions comprising an anti-viral drug and a small molecule inhibitor of class I and/or class IIb HDACs. Exemplary anti-viral drugs include nirmatrelvir and ritonavir (Paxlovid); sotrovimab (Xevudy); remdesivir (Veklury); and molnupiravir (Lagevrio), and/or an antibody therapy, e.g., bebtelovimab. Such compositions are particularly useful for treating viral infections, e.g., infection with a coronavirus such as SARS-COV-2.

Pharmaceutical compositions are typically formulated to be compatible with its intended route of administration. Examples of routes of administration include parenteral, e.g., intravenous, intradermal, subcutaneous, oral, nasal (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the examples below.

### Experimental Model and Subject Details

### Animals

6-8 week-old female C57BL/6J (Jax 000664) and C57BL/6J-Hdac6em2Lutzy/J (Jax 029318) mice were purchased from The Jackson Laboratory. Mice were housed under specific pathogen-free conditions at Boston Children's Hospital, and all the procedures were approved under the Institutional Animal Care and Use Committee (IACUC) and operated under the supervision of the department of Animal Resources at Children's Hospital (ARCH). Sex as a biological variable was not analyzed in this work.

### Neutrophil Purification

Human neutrophils from blood of healthy donors were purified with Polymorphoprep (Progen) according to manufacturer's instruction. Murine neutrophils were purified over a 62.5% Percoll gradient (GE Healthcare) as previously described (Broggi et al., 2017). Purified neutrophils were resuspended in DMEM medium (ThermoFisher) and plated in a 96 multi-well plate previously coated with poly-D-lysine (ThermoFisher). Plate was spin at 200×g for 1 minute without brake to facilitate neutrophils seeding.

### Methods Details

### Extracellular DNA Quantification

Neutrophils were treated with indicated drugs for 1 hour and then stimulated with 1 μM PMA for 3 hours. Extracellular DNA was quantified in the supernatant with Quant-iT PicoGreen (Invitrogen).

### Citrullinated Histone H3 In Vitro Quantification and Imaging

Neutrophils pretreated with indicated HDAC inhibitors, or protein-arginine deaminase (PAD4) inhibitor GSK484, and then stimulated with 1 μM PMA for 3 hours, were fixed with equal volume of 10% formalin for 1 h at room temperature. Samples were blocked with 2% BSA in PBS for 1 hour at room temperature and stained with the anti-citrullinated histone H3 antibody overnight at 4° C., followed by anti-rabbit Alexa 568 secondary antibody. Nuclei were stained with DAPI. NETs formation was measured as induction of citrullinate histone H3 positivity and quantified with a plate-reader (Ex 568 nm, Em 603 nm). Percentage of NET induction was calculated using signal in un-treated cells as 0% of induction and signal in PMA-treated cells as 100% of induction. Alternatively, NETosis induction was quantified by imaging. Images were acquired with EVOS M7000 Imaging System (ThermoFisher Scientific). Number of citrullinated histone H3 positive cells and area of DAPI signal were quantified in multiple fields of view (FOV) using ImageJ software (NIH). For 3D nuclei reconstruction images were acquired with ZEISS 880 Fast Airyscan and analyzed with ZEISS ZEN Blue Software.

### Immunoblotting

For western blotting, 2×10⁶ neutrophils pretreated with indicated HDAC inhibitor, or protein-arginine deaminase (PAD4) inhibitor GSK484, and then stimulated with 1 μM PMA for 3 hours were lysed using RIPA buffer with protease and phosphatase inhibitors (T-2494, A.G. Scientific) and diisopropylfluorophosphate (D0879, Sigma). Immunoblotting was performed using standard molecular biology techniques. Band intensity was quantified using ImageJ software.

### ROS Quantification

Neutrophils plated in a white 96 multi-well plate were pretreated with indicated HDAC inhibitor, or protein-arginine deaminase (PAD4) inhibitor GSK484 for 1 hour and then were stimulated with 1 μM PMA in presence of luminol (Cat #A8511, Sigma) and HRP (Sigma). ROS production was monitored in real-time by luminescence. Luminescence was measured every 3 minutes for 3 hours in a plate-reader analyzer (Molecular Device).

### Phagocytosis Assay

E. coli (O111) was stained with Cell Proliferation Dye eFluor™ 670 (cat #65-0840-85, Invitrogen) for 30 minutes and then opsonized with 20% mouse serum for 10 minutes. Neutrophils were stimulated with the indicated MOI for 1 hour at 37° C., washed, and then stained with anti-Ly6G PE antibody and DAPI. Ultimately, samples were acquired with BD FACS Fortessa and percentage of neutrophils positive for E. coli was quantified.

### In Vivo NET Quantification

NETosis was measured in bronco-alveolar lavage fluid or lung tissue homogenates as quantity of citrullinated histone H3 with Citrullinated histone H3 ELISA kit (Cayman) and as quantity of myeloperoxidase (MPO)-DNA complexes by using a modified Cell Death Detection ELISA (Roche). Briefly, anti-myeloperoxidase antibody (ThermoFisher) was used to coat a high-binding 96 multi-well plate overnight at 4° C. Following a 1 h blocking with 2% BSA/PBS solution, samples were added for an overnight incubation at 4° C. Finally, DNA was detected with Cell Death Detection kit according to manufacture instruction. Absorbance was measured at 405 nm and MPO-DNA complex levels were quantified as normalized relative to saline.

Alternatively, neutrophils positive for citrullinated histone H3 were quantified by cytofluorimetry. Briefly, neutrophils from bronco-alveolar lavage fluid or peritoneal lavage fluid were stained for Ly6G, CD11b, CD45 and Zombie dye for 20 minutes at room temperature and then fixed with BD FACS Lysing Solution (cat #349202, BD Biosciences). Samples were washed twice with Intracellular Staining Permeabilization Wash buffer (cat #421002, Biolegend), incubated with anti-citrullinated histone H3 antibody (Abcam) 1 hour at room temperature and subsequently with PE anti-rabbit antibody for 30 minutes. Finally, samples were acquired with BD FACS Fortessa and neutrophils (CD45+/Ly6G+/CD11b+) positive for citrullinated histone H3 were quantified with FlowJo software.

### Poly(I:C) Pulmonary NET Induction

6-8 weeks-old female mice were treated daily with 2.5 mg/kg of poly (I:C) for 4 days and euthanized 18 hours post the last poly(I:C) intra-tracheal instillation, as previously described (Broggi et al., 2020). Temperatures were monitored with a rectal probe. NETosis was measured in bronco-alveolar lavage fluid and lung homogenates as quantity of citrullinated histone H3 and quantity of MPO-DNA complexes. Ricolinostat (30 mg/kg/dose) and GSK484 (25 mg/kg/dose) were administered intraperitoneally at day 2, day 4 and 3 hours prior the end point. To assess lung permeability, albumin in BALF was measured with Albumin Assay Kit (ab235628, Abcam). Number of neutrophils (CD45+/Ly6G+/CD11b+) in blood, BALF and lung homogenate was quantified by cytofluorimetry using CountBright Absolute Cell Counting Beads (Thermo Scientific). Paraffin-embedded lung sections were stained with DAPI for nuclei and with anti-neutrophil antibody (Abcam) for the presence of neutrophils.

### Measurement of Airway Functional Responses

Airway hyperreactivity (AHR) was measured, as previously described (Harb et al., 2020). Anesthetized mice were exposed to doubling concentrations of aerosolized acetyl-β-methacholine (Sigma-Aldrich) by using a Buxco small-animal ventilator (Data Sciences International). The relative peak airway resistance for each methacholine dose, normalized to the saline baseline (Score=1), was calculated.

S. aureus Pulmonary Infection Model

6-8-week-old female mice were pretreated with indicated HDAC inhibitors at −1 day and −3 hours prior in vivo NETs induction by intra-tracheal instillation of 65×10⁶ CFU of Staphylococcus aureus subsp. aureus Rosenbach (ATCC 25904). Ricolinostat was used at 30 mg/kg/dose and GSK484 at 25 mg/kg/dose. Temperatures were monitored with a rectal probe. NETosis was measured in bronco-alveolar lavage fluid as quantity of citrullinated histone H3 and quantity of MPO-DNA complexes by ELISA. Alternatively, neutrophils positive for citrullinated histone H3 were analyzed by cytofluorimetry in BALFs samples. Albumin in BALF was measured with Albumin Assay Kit (ab235628, Abcam). Number of neutrophils (CD45+/Ly6G+/CD11b+) in blood and BALF was quantified by flow cytometry using CountBright Absolute Cell Counting Beads (Thermo Scientific).

### In Vivo LPS-Induced Septic Shock

8-week-old female mice were primed with 1 mg/kg of LPS intra peritoneum (i.p.) for 5 hours and then challenged with 2 mg/kg of LPS i.p. Ricolinostat was used at 30 mg/kg/dose, GSK484 at 25 mg/kg/dose and dexamethasone at 10 mg/kg/dose. Drugs were administered i.p. at day −1 and 2 hours prior priming. Blood samples were collected 2 hours post-challenge and cytokines were measured by ELISA. The platelet concentrations and neutrophils numbers in the blood and peritoneal cavity were calculated by cytofluorimetry using CountBright Absolute Cell Counting Beads (Thermo Scientific) according to the manufacturer's instructions. NETosis was measured as quantity of neutrophils in the peritoneal lavage positive for citrullinated histone H3 by cytofluorimetry.

### Quantification and Statistical Analysis

Results were analyzed with GraphPad Prism statistical software (version 8). One-way ANOVA and Two-way ANOVA were used to analyze statistically significant differences between the means of two or more independent groups, as indicated in the figure legends. Sample sizes for each experiment are provided in the figures and the respective legends. Asterisks were used as follows: *p<0.05, **p<0.01, ***p<0.001 and ****p<0.0001.

### Example 1. Class I/IIb HDACs Drive NET Formation

To test the capacity of HDACs to modulate NET formation, we assembled a focused collection of class and isoform specific inhibitors of HDACs with high structural and functional diversity (Park and Kim, 2020). This included pan-HDAC inhibitors that selectively target HDAC class I (HDAC1/2/3/8), class IIa (HDAC4/5/7/9), class IIb (HDAC6/10), and class III (sirtuins), as well as an inhibitor targeting bromodomain proteins, which recognize acetylated lysine on histones. Initially, we investigated the capacity of these inhibitors to alter NETosis by measuring extracellular DNA release by primary human neutrophils activated with phorbol myristate acetate (PMA), a well-known inducer of NETs. DNA release is often used as a proxy for NETosis and, indeed, inhibition of NET induction by using the PAD4 inhibitor GSK484 resulted in significant decrease of extracellular DNA by PMA-treated cells (FIG. 5A). Compounds that inhibited either class I or class I/IIb HDACs also significantly diminished extracellular DNA secretion from PMA-treated neutrophils (FIG. 5B), whereas class IIa, pan-HDAC inhibitors, other HDACs inhibitors, or bromodomain inhibition did not affect DNA release. These data demonstrate that inhibition of certain, but not all, classes of HDACs efficiently decreases the release of extracellular DNA from PMA-treated human neutrophils and suggest that targeting HDACs may effectively prevent NETosis. Thus, we focused on the compounds that significantly reduced DNA release to further assess their activity on NETosis. To confirm the capacity of specific HDAC inhibitors to interfere with NETosis, we investigated whether HDAC inhibition also prevented the release of citrullinated histone H3 (CitH3), a hallmark of NET formation (Thiam et al., 2020; Wang et al., 2009), that peaked 3-5 hours upon PMA administration (FIG. 5C). In agreement with previous findings (Lewis et al., 2015), pretreatment with the NET-disrupting PAD4 inhibitor GSK484 dramatically reduced NET induction, measured as CitH3 staining, in PMA-stimulated human neutrophils (FIGS. 1A and 5D). Similar to extracellular DNA release, class I or class I/II HDAC inhibitors entinostat and ricolinostat prevented NET release in a dose-dependent manner with an efficacy comparable with GSK484 (FIGS. 1A and 5D). Next, we tested if HDACs regulate NETosis not only in human but also in mouse neutrophils. We found that, similarly to what we observed using human cells, the class I/IIb HDAC inhibitor ricolinostat efficiently decreased the release of CitH3 in PMA-treated mouse neutrophils (FIG. 1B). In contrast to the human cell data, the class I HDAC-specific inhibitor entinostat had no effect on murine neutrophils. In keeping with a key role of class I/II HDACs in regulating NET release, we also found that ricolinostat significantly reduced the expansion of nuclei, another hallmark of NETosis in PMA-treated cells (Sollberger et al., 2018) (FIGS. 1C and 1D). Moreover, ricolinostat inhibited NET induction in response to nigericin, a bacterial toxin known to induce NETosis in neutrophils (Chen et al., 2018; Sollberger et al., 2018) (FIG. 5E). Overall, these data imply that zinc-dependent lysine deacetylases represented by class I/IIb HDACs are required to induce NETosis both in human and mouse neutrophils, and that the class I/IIb HDAC inhibitor ricolinostat, a phase II clinical trial drug, efficiently inhibits NETosis both in humans and mouse neutrophils.

A previous study suggested that pan-HDAC inhibition switches neutrophil cell death from NETosis to apoptosis (Hamam and Palaniyar, 2019). To assess whether ricolinostat reduced NETosis by favoring other types of cell death, we tested the levels of apoptosis, pyroptosis, and/or necrosis induction in PMA-treated cells exposed to either ricolinostat or GSK484. We excluded the activation of apoptosis or necroptosis under our experimental conditions (FIG. 1E). In agreement with previous reports (Chen et al., 2018; Sollberger et al., 2018), we found that PMA induced gasdermin D (GSDMD) cleavage, but, notably, this process was not altered by either ricolinostat or GSK484 (FIG. 1E). Overall, these data confirm the capacity of ricolinostat, as well as GSK484, to prevent NETosis without affecting the induction of other forms of cell death.

Next, we assessed how HDAC inhibition prevents NETosis. Given that inhibition of class I/IIb HDACs decreased the release of citrullinated histone H3, we hypothesized that ricolinostat exerts its functions by increasing H3 acetylation, which in turn may prevent PAD4-dependent citrullination. Indeed, our data demonstrated that, upon ricolinostat, but not GSK484, administration, histone H3 acetylation was boosted in untreated as well as PMA-treated cells (FIGS. 1E and 1F). When cells were treated with PMA, and citrullination was increased to induce NETosis, ricolinostat significantly decreased H3 citrullination compared with cells that were treated with PMA only. As expected, the PAD4-inhibitor GSK484 completely abrogated H3 citrullination (FIGS. 1E and 1F). In keeping with the capacity of ricolinostat to affect neutrophil responses by preventing histone deacetylation, we found that ricolinostat treatment did not alter reactive oxygen species (ROS) production, another key step in NETosis (Douda et al., 2015; Fuchs et al., 2007; Remijsen et al., 2011), either in human or mouse cells (FIG. 1G). Similarly, we excluded that ricolinostat administration altered other important functions of neutrophils, such as bacterial phagocytosis (FIG. 1H). These data support that the capacity of ricolinostat to prevent histone H3 deacetylation impedes its citrullination and the release of NETs. Therefore, our data support a model in which acetylation and citrullination modifications on histone H3 are divergently regulated in the context of NETosis.

We next focused on the specificity of action of ricolinostat. Ricolinostat inhibits both class I and class IIb HDACs, with a preference for the latter. We, thus, compared the activity of ricolinostat with other inhibitors specific for class IIb HDACs (tubastatin A and nexturastat) as well as with tubacin, that similarly to ricolinostat targets not only class IIb HDACs but also class I HDACs (Bergman et al., 2012; Butler et al., 2010; Haggarty et al., 2003). The siderophore deferoxamine and the hydroxamate-based metalloprotease inhibitor batimastat were used as negative controls to exclude bystander activities of the drugs utilized on other divalent metals. We found that class I/IIb HDAC inhibition prevented NETosis, whereas selective class IIb targeting did not (FIG. 5F). In agreement with these data, neutrophils that lack HDAC6, the major class IIb HDAC, behaved similarly to wild-type cells and remained sensitive to ricolinostat administration (FIG. 5G). Finally, human neutrophils derived from two independent donors as well as murine neutrophils were treated, or not, with ricolinostat or its chlorinated analog citarinostat (or GSK484, used as a positive control) and the level of CitH3 was assessed. Our data revealed both compounds inhibited NET formation to a comparable extent (FIGS. 5H-5J). Overall, these data demonstrate that the capacity of zinc-dependent class I/IIb HDACs to drive NET formation is conserved in humans and mice.

### Example 2. Inhibition of Class I/IIb HDACs In Vivo Dampens NETosis and Protects Against Pneumonia Induced by Exposure to Viral Ligands

The conserved activity of class I/IIb HDACs in regulating NETosis in human and mouse cells prompted us to assess how HDAC inhibition with ricolinostat affects the development of pathological inflammation in vivo in murine models. To determine if the capacity of ricolinostat to inhibit NETosis as assessed in vitro also impacts viral pneumonia, we used a well-characterized mouse model that mimics the immune response to RNA respiratory viruses and that we previously successfully used to unveil some features of RNA viral infections (Broggi et al., 2020). The synthetic analog of double-stranded RNA polyinosine: polycytidylic acid (poly(I:C)) was intratracheally instilled to mice daily for 4 days to induce NET formation (FIG. 6A). Poly I:C was administered in the presence or absence of ricolinostat, and PAD4 inhibition with GSK484 was used as a comparison (FIG. 2A). Administration of either ricolinostat or GSK484 significantly reduced morbidity in poly(I:C)-treated mice, measured as temperature drop (FIG. 2B). Most importantly, lung permeability and functionality, measured as previously described in mouse models based on the use of synthetic viral ligands and/or influenza A viral infection (Broggi et al., 2020; Harb et al., 2020, Harb et al., 2021; Jamieson et al., 2013), were significantly preserved in mice that received either ricolinostat or GSK484 and poly(I:C), compared with mice that were treated with poly(I:C) only (FIGS. 2C and 2D). Poly(I:C)-treated mice showed a significant increase of NETosis in the bronco-alveolar lavage fluid (BALF) and in the lung tissue, measured as levels of CitH3 (FIGS. 2E and 2G), as well as by levels of myeloperoxidase (MPO)-DNA complexes (FIGS. 2F and 2H). Increased NET induction was abrogated upon drug treatment (FIGS. 2E-2H). Although neutrophil levels in the blood did not change significantly among different treatments (FIGS. 6B and 6C), in keeping with a reduced death of neutrophils by NETosis, we found a trend of accumulation of neutrophils in the BALF as well as in the lung of mice treated with ricolinostat and GSK484 compared with mice administered poly(I:C) only (FIGS. 6D-6F). Of note, in all the conditions tested, ricolinostat phenocopied, and in some cases outcompeted (i.e., increased survival of neutrophils in the BALF), the activity of GSK484. These results demonstrate that class I/IIb HDACs play a role as important as the key enzyme PAD4 in regulating NETosis and that zinc-dependent HDACs can be targeted against viral-induced ARDS.

### Example 3. Class I/IIb HDACs Increase NETosis and ARDS in a Mouse Model of Bacterial Pneumonia

To further assess whether the capacity of ricolinostat to prevent NETosis can be used against ARDS, we employed a bacterial lung pneumonia mouse model based on the intratracheal injection of S. aureus (FIG. 3A). As before, in this set of experiments we compared the activity of ricolinostat with the PAD4 inhibitor GSK484. When class I/IIb HDACs were inhibited with ricolinostat, morbidity was significantly decreased compared with mice injected with S. aureus only, and also with S. aureus and GSK484 (FIG. 3B). Lung barrier alteration was prevented both by ricolinostat or GSK484 treatment, compared with mice exposed to S. aureus alone, whereas bacterial control was not affected by these treatments (FIGS. 3C and 3D). These data suggest the capacity of these drugs to affect tissue tolerance without changing immune resistance (Medzhitov et al., 2012). NETs are well known to alter tissue functionality and, in fact, have been shown to dampen tissue tolerance during lung infections (Iwasaki and Pillai, 2014; Pillai et al., 2016). Indeed, we found that administration of either ricolinostat or GSK484 potently decreased NETosis as measured by levels of citrullinated histone H3 as well as MPO-DNA complexes in the BALF of mice treated with S. aureus (FIGS. 3E and 3F). The capacity of these drugs to inhibit NETosis was further demonstrated by cytofluorimetric quantification of citrullinated histone H3-positive neutrophils in the BALF (FIG. 3G). In keeping with our previous findings in the poly(I:C)-driven pneumonia model, we found no major differences in the levels of neutrophils circulating in the blood, whereas reduction of NETosis led to the presence of increased numbers of neutrophils in the BALF (FIGS. 3H and 3I, and 7A). Pro-inflammatory cytokines IL-6 and IL-1β, but not TNF, were also significantly decreased in the BALF of mice administered ricolinostat and GSK484, compared with mice that received S. aureus only (FIGS. 7B-7D). Collectively, these results demonstrate that zinc-dependent HDAC inhibition is as efficacious as PAD4 inhibition in preventing bacterial-induced NETosis and ARDS. Also, treatment with ricolinostat was more efficient in terms of morbidity reduction compared with GSK484.

### Example 4. Class I/IIb HDAC Inhibition Protects Against Systemic Inflammation

Finally, we investigated whether inhibition of class I/IIb HDACs dampens systemic inflammation in a mouse model of septic shock. Septic shock is characterized by systemic inflammation and disseminated intravascular coagulation (van der Poll et al., 2017), and neutrophils play a critical role in driving this lethal syndrome. We hypothesized that inhibition of NET formation using ricolinostat may yield beneficial effects in reducing sepsis-associated symptoms. Thus, we employed a well-established lipopolysaccharide (LPS)-driven model of septic shock (Zanoni et al., 2017) in mice treated, or not, with ricolinostat or GSK484, as a comparison (FIG. 4A). Mice that received either ricolinostat or GSK484 showed no temperature loss, compared with untreated animals, and were significantly protected compared with mice treated with LPS only (FIG. 4B). Previous studies demonstrated that decreased platelet counts are a hallmark of disseminated intravascular coagulation during sepsis (Yang et al., 2019). It is also worth noting that NETs are major drivers of disseminated coagulation in sepsis (Xu et al., 2009) and that recent studies underscore that interactions between platelets and neutrophils are critical in sustaining disseminated intravascular coagulation (Clark et al., 2007; McDonald et al., 2012, McDonald et al., 2017). Of note, platelet counts were significantly higher in mice that received ricolinostat or GSK484, compared with mice treated with LPS alone (FIG. 4C). Although more data will be needed to directly link HDAC inhibition to the reduction of disseminated coagulation during septic shock, these data suggest that immunothrombosis can be similarly prevented upon administration of inhibitors that target either HDAC or PAD4. Indeed, also in this septic shock mouse model we confirmed the efficacy of class I/IIb HDAC or PAD4 inhibition in preventing NETosis. We found not only reduced release of NETs by neutrophils in the peritoneal lavage (FIG. 4D) but also increased neutrophils levels (FIGS. 8A-8C). As before, these data confirm the efficacy of the used drugs in preventing NETosis and death of neutrophils. In keeping with the systemic nature of this sepsis model, and with the positive feedback loop recently described between NETosis and increased cytokine production (Apel et al., 2021), we found that NET inhibition by either ricolinostat or GSK484 significantly decreased pro-inflammatory cytokine production (FIGS. 4E-4G).

Since dexamethasone is widely used to dampen inflammation, we also compared the therapeutic effects of ricolinostat with dexamethasone. We did not observe a significant difference in platelet numbers when comparing the dexamethasone-treated group with the group treated with LPS only (FIG. 8D), whereas both drugs efficiently reduced pro-inflammatory cytokine production (FIGS. 8E and 8F). Overall, our results demonstrate that inhibition of NETosis by ricolinostat protects against systemic inflammation in a mouse model of septic shock.

### REFERENCES

Ackermann, M., Verleden, S.E., Kuehnel, M., Haverich, A., Welte, T., Laenger, F., Vanstapel, A., Werlein, C., Stark, H., Tzankov, A., et al. (2020). Pulmonary Vascular Endothelialitis, Thrombosis, and Angiogenesis in Covid-19. New Engl J Med 383, 120-128.
Al-Samkari, H., Karp Leaf, R.S., Dzik, W.H., Carlson, J.C.T., Fogerty, A.E., Waheed, A., Goodarzi, K., Bendapudi, P.K., Bornikova, L., Gupta, S., et al. (2020). COVID-19 and coagulation: bleeding and thrombotic manifestations of SARS-COV-2 infection. Blood 136, 489-500.
Apel, F., Andreeva, L., Knackstedt, L.S., Streeck, R., Frese, C.K., Goosmann, C., Hopfner, K.P., and Zychlinsky, A. (2021). The cytosolic DNA sensor cGAS recognizes neutrophil extracellular traps. Sci Signal 14, eaax7942.
Arpinati, L., Shaul, M.E., Kaisar-Iluz, N., Mali, S., Mahroum, S., and Fridlender, Z.G. (2020). NETosis in cancer: a critical analysis of the impact of cancer on neutrophil extracellular trap (NET) release in lung cancer patients vs. mice. Cancer Immunol Immunother 69, 199-213.

Bergman, J.A., Woan, K., Perez-Villarroel, P., Villagra, A., Sotomayor, E.M., and Kozikowski, A.P. (2012). Selective Histone Deacetylase 6 Inhibitors Bearing Substituted Urea Linkers Inhibit Melanoma Cell Growth. J Med Chem 55, 9891-9899.
Blondonnet, R., Constantin, J.-M., Sapin, V., and Jabaudon, M. (2016). A Pathophysiologic Approach to Biomarkers in Acute Respiratory Distress Syndrome. Dis Markers 2016, 3501373.
Brinkmann, V., Reichard, U., Goosmann, C., Fauler, B., Uhlemann, Y., Weiss, D.S., Weinrauch, Y., and Zychlinsky, A. (2004). Neutrophil Extracellular Traps Kill Bacteria. Science 303, 1532-1535.
Broggi, A., Ghosh, S., Sposito, B., Spreafico, R., Balzarini, F., Lo Cascio, A., Clementi, N., De Santis, M., Mancini, N., Granucci, F., et al. (2020). Type III interferons disrupt the lung epithelial barrier upon viral recognition. Science 369, 706-712.

Broggi, A., Tan, Y., Granucci, F., and Zanoni, I. (2017). IFN-lambda suppresses intestinal inflammation by non-translational regulation of neutrophil function. Nat Immunol 18, 1084-1093.
Butler, K.V., Kalin, J., Brochier, C., Vistoli, G., Langley, B., and Kozikowski, A.P. (2010). Rational Design and Simple Chemistry Yield a Superior, Neuroprotective HDAC6 Inhibitor, Tubastatin A. J Am Chem Soc 132, 10842-10846.
Channappanavar, R., and Perlman, S. (2017). Pathogenic human coronavirus infections: causes and consequences of cytokine storm and immunopathology. Semin Immunopathol 39, 529-539.
Chen, K.W., Monteleone, M., Boucher, D., Sollberger, G., Ramnath, D., Condon, N.D., von Pein, J.B., Broz, P., Sweet, M.J., and Schroder, K. (2018). Noncanonical inflammasome signaling elicits gasdermin D-dependent neutrophil extracellular traps. Sci Immunol 3, eaar6676.
Clark, S.R., Ma, A.C., Tavener, S.A., McDonald, B., Goodarzi, Z., Kelly, M.M., Patel, K.D., Chakrabarti, S., McAvoy, E., Sinclair, G.D., et al. (2007). Platelet TLR4 activates neutrophil extracellular traps to ensnare bacteria in septic blood. Nat Med 13, 463-469.
Douda, D.N., Khan, M.A., Grasemann, H., and Palaniyar, N. (2015). SK3 channel and mitochondrial ROS mediate NADPH oxidase-independent NETosis induced by calcium influx. Proc Natl Acad Sci U S A 112, 2817-2822.
Fuchs, T.A., Abed, U., Goosmann, C., Hurwitz, R., Schulze, I., Wahn, V., Weinrauch, Y., Brinkmann, V., and Zychlinsky, A. (2007). Novel cell death program leads to neutrophil extracellular traps. J Cell Biol 176, 231-241.
Giannis, D., Ziogas, I.A., and Gianni, P. (2020). Coagulation disorders in coronavirus infected patients: COVID-19, SARS-COV-1, MERS-COV and lessons from the past. J Clin Virol 127, 104362.
Gomez-Moreno, D., Adrover, J.M., and Hidalgo, A. (2018). Neutrophils as effectors of vascular inflammation. Eur J Clin Invest 48 Suppl 2, e12940.
Grunstein, M. (1997). Histone acetylation in chromatin structure and transcription. Nature 389, 349-352.
Guan, W.-j., Ni, Z.-y., Hu, Y., Liang, W.-h., Ou, C.-q., He, J.-x., Liu, L., Shan, H., Lei, C.-1., Hui, D.S.C., et al. (2020). Clinical Characteristics of Coronavirus Disease 2019 in China. New Engl J Med 382, 1708-1720.
Haggarty, S.J., Koeller, K.M., Wong, J.C., Grozinger, C.M., and Schreiber, S.L. (2003). Domain-selective small-molecule inhibitor of histone deacetylase 6 (HDAC6)-mediated tubulin deacetylation. Proc Natl Acad Sci USA 100, 4389.
Hahn, J., Schauer, C., Czegley, C., Kling, L., Petru, L., Schmid, B., Weidner, D., Reinwald, C., Biermann, M.H.C., Blunder, S., et al. (2019). Aggregated neutrophil extracellular traps resolve inflammation by proteolysis of cytokines and chemokines and protection from antiproteases. FASEB J 33, 1401-1414.
Hamam, H.J., Khan, M.A., and Palaniyar, N. (2019). Histone Acetylation Promotes Neutrophil Extracellular Trap Formation. Biomolecules 9, 32.
Hamam, H.J., and Palaniyar, N. (2019). Histone Deacetylase Inhibitors Dose-Dependently Switch Neutrophil Death from NETosis to Apoptosis. Biomolecules 9.
Harb, H., Benamar, M., Lai, P.S., Contini, P., Griffith, J.W., Crestani, E., Schmitz-Abe, K., Chen, Q., Fong, J., Marri, L., et al. (2021). Notch4 signaling limits regulatory T-cell-mediated tissue repair and promotes severe lung inflammation in viral infections. Immunity.
Harb, H., Stephen-Victor, E., Crestani, E., Benamar, M., Massoud, A., Cui, Y., Charbonnier, L.M., Arbag, S., Baris, S., Cunnigham, A., et al. (2020). A regulatory T cell Notch4-GDF15 axis licenses tissue inflammation in asthma. Nat Immunol 21, 1359-1370.
Iba, T., Levy, J.H., Raj, A., and Warkentin, T.E. (2019). Advance in the Management of Sepsis-Induced Coagulopathy and Disseminated Intravascular Coagulation. J Clin Med 8.
Iwasaki, A., and Pillai, P.S. (2014). Innate immunity to influenza virus infection. Nat Rev Immunol 14, 315-328.
Jaillon, S., Galdiero, M.R., Del Prete, D., Cassatella, M.A., Garlanda, C., and Mantovani, A. (2013). Neutrophils in innate and adaptive immunity. Semin Immunopathol 35, 377-394.
Jamieson, A.M., Pasman, L., Yu, S., Gamradt, P., Homer, R.J., Decker, T., and Medzhitov, R. (2013). Role of tissue protection in lethal respiratory viral-bacterial coinfection. Science 340, 1230-1234.
Kang, L., Yu, H., Yang, X., Zhu, Y., Bai, X., Wang, R., Cao, Y., Xu, H., Luo, H., Lu, L., et al. (2020). Neutrophil extracellular traps released by neutrophils impair revascularization and vascular remodeling after stroke. Nat Commun 11, 2488.
Kenny, E.F., Herzig, A., Krüger, R., Muth, A., Mondal, S., Thompson, P.R., Brinkmann, V., Bernuth, H.v., and Zychlinsky, A. (2017). Diverse stimuli engage different neutrophil extracellular trap pathways. eLife 6, e24437.
Lee, J.S., Park, S., Jeong, H.W., Ahn, J.Y., Choi, S.J., Lee, H., Choi, B., Nam, S.K., Sa, M., Kwon, J.S., et al. (2020). Immunophenotyping of COVID-19 and influenza highlights the role of type I interferons in development of severe COVID-19. Sci Immunol 5, eabd1554.
Lee, K.H., Kronbichler, A., Park, D.D., Park, Y., Moon, H., Kim, H., Choi, J.H., Choi, Y., Shim, S., Lyu, I.S., et al. (2017). Neutrophil extracellular traps (NETs) in autoimmune diseases: A comprehensive review. Autoimmun Rev 16, 1160-1173.
Lewis, H.D., Liddle, J., Coote, J.E., Atkinson, S.J., Barker, M.D., Bax, B.D., Bicker, K.L., Bingham, R.P., Campbell, M., Chen, Y.H., et al. (2015). Inhibition of PAD4 activity is sufficient to disrupt mouse and human NET formation. Nat Chem Biol 11, 189-191.
Lucas, C., Wong, P., Klein, J., Castro, T.B.R., Silva, J., Sundaram, M., Ellingson, M.K., Mao, T., Oh, J.E., Israelow, B., et al. (2020). Longitudinal analyses reveal immunological misfiring in severe COVID-19. Nature 584, 463-469.
Magupalli, V.G., Negro, R., Tian, Y., Hauenstein, A.V., Di Caprio, G., Skillern, W., Deng, Q., Orning, P., Alam, H.B., Maliga, Z., et al. (2020). HDAC6 mediates an aggresome-like mechanism for NLRP3 and pyrin inflammasome activation. Science 369, eaas8995.
McDonald, B., Davis, R.P., Kim, S.J., Tse, M., Esmon, C.T., Kolaczkowska, E., and Jenne, C.N. (2017). Platelets and neutrophil extracellular traps collaborate to promote intravascular coagulation during sepsis in mice. Blood 129, 1357-1367.
McDonald, B., Urrutia, R., Yipp, B.G., Jenne, C.N., and Kubes, P. (2012). Intravascular neutrophil extracellular traps capture bacteria from the bloodstream during sepsis. Cell Host Microbe 12, 324-333.
Medzhitov, R., Schneider, D.S., and Soares, M.P. (2012). Disease tolerance as a defense strategy. Science 335, 936-941.
Mehta, P., McAuley, D.F., Brown, M., Sanchez, E., Tattersall, R.S., Manson, J.J., and Hlh Across Speciality Collaboration, U.K. (2020). COVID-19: consider cytokine storm syndromes and immunosuppression. Lancet 395, 1033-1034.
Middleton, E.A., He, X.-Y., Denorme, F., Campbell, R.A., Ng, D., Salvatore, S.P., Mostyka, M., Baxter-Stoltzfus, A., Borczuk, A.C., Loda, M., et al. (2020). Neutrophil extracellular traps contribute to immunothrombosis in COVID-19 acute respiratory distress syndrome. Blood 136, 1169-1179.
Nathan, C. (2002). Points of control in inflammation. Nature 420, 846-852.
Papayannopoulos, V., Metzler, K.D., Hakkim, A., and Zychlinsky, A. (2010). Neutrophil elastase and myeloperoxidase regulate the formation of neutrophil extracellular traps. J Cell Biol 191, 677-691.
Park, S.-Y., and Kim, J.-S. (2020). A short guide to histone deacetylases including recent progress on class II enzymes. Exp Mol Med 52, 204-212.
Perlman, S., and Dandekar, A.A. (2005). Immunopathogenesis of coronavirus infections: implications for SARS. Nat Rev Immunol 5, 917-927.
Pillai, P.S., Molony, R.D., Martinod, K., Dong, H., Pang, I.K., Tal, M.C., Solis, A.G., Bielecki, P., Mohanty, S., Trentalange, M., et al. (2016). Mx1 reveals innate pathways to antiviral resistance and lethal influenza disease. Science 352, 463-466.
Porto, B.N., and Stein, R.T. (2016). Neutrophil Extracellular Traps in Pulmonary Diseases: Too Much of a Good Thing? Front Immunol 7, 311.
Radermecker, C., Detrembleur, N., Guiot, J., Cavalier, E., Henket, M., d′Emal, C., Vanwinge, C., Cataldo, D., Oury, C., Delvenne, P., et al. (2020). Neutrophil extracellular traps infiltrate the lung airway, interstitial, and vascular compartments in severe COVID-19. J Exp Med 217, e20201012.
Remijsen, Q., Vanden Berghe, T., Wirawan, E., Asselbergh, B., Parthoens, E., De Rycke, R., Noppen, S., Delforge, M., Willems, J., and Vandenabeele, P. (2011). Neutrophil extracellular trap cell death requires both autophagy and superoxide generation. Cell Res 21, 290-304.
Ruan, Q., Yang, K., Wang, W., Jiang, L., and Song, J. (2020). Clinical predictors of mortality due to COVID-19 based on an analysis of data of 150 patients from Wuhan, China. Intensive Care Med 46, 846-848.
Saitoh, T., Komano, J., Saitoh, Y., Misawa, T., Takahama, M., Kozaki, T., Uehata, T., Iwasaki, H., Omori, H., Yamaoka, S., et al. (2012). Neutrophil Extracellular Traps Mediate a Host Defense Response to Human Immunodeficiency Virus-1. Cell Host Microbe 12, 109-116.
Schauer, C., Janko, C., Munoz, L.E., Zhao, Y., Kienhofer, D., Frey, B., Lell, M., Manger, B., Rech, J., Naschberger, E., et al. (2014). Aggregated neutrophil extracellular traps limit inflammation by degrading cytokines and chemokines. Nature medicine 20, 511-517.
Seto, E., and Yoshida, M. (2014). Erasers of histone acetylation: the histone deacetylase enzymes. Cold Spring Harb Perspect Biol 6, a018713-a018713.
Shin, H.-S., Kim, Y., Kim, G., Lee, J.Y., Jeong, I., Joh, J.-S., Kim, H., Chang, E., Sim, S.Y., Park, J.-S., et al. (2019). Immune Responses to Middle East Respiratory Syndrome Coronavirus During the Acute and Convalescent Phases of Human Infection. Clin Infect Dis 68, 984-992.
Short, K.R., Kroeze, E.J.B.V., Fouchier, R.A.M., and Kuiken, T. (2014). Pathogenesis of influenza-induced acute respiratory distress syndrome. Lancet Infect Dis 14, 57-69.
Sollberger, G., Choidas, A., Burn, G.L., Habenberger, P., Di Lucrezia, R., Kordes, S., Menninger, S., Eickhoff, J., Nussbaumer, P., Klebl, B., et al. (2018). Gasdermin D plays a vital role in the generation of neutrophil extracellular traps. Sci Immunol 3, eaar6689.
Tang, B.M., Cootes, T., and McLean, A.S. (2019). From Influenza-Induced Acute Lung Injury to Multiorgan Failure. In Annual Update in Intensive Care and Emergency Medicine 2019, J.-L. Vincent, ed. (Cham: Springer International Publishing), pp. 449-458.
Tang, N., Li, D., Wang, X., and Sun, Z. (2020). Abnormal coagulation parameters are associated with poor prognosis in patients with novel coronavirus pneumonia. J Thromb Haemost 18, 844-847.
Thiam, H.R., Wong, S.L., Wagner, D.D., and Waterman, C.M. (2020). Cellular Mechanisms of NETosis. Annu Rev Cell Dev Biol 36, 191-218.
van der Poll, T., van de Veerdonk, F.L., Scicluna, B.P., and Netea, M.G. (2017). The immunopathology of sepsis and potential therapeutic targets. Nat Rev Immunol 17, 407-420.
Veras, F.P., Pontelli, M.C., Silva, C.M., Toller-Kawahisa, J.E., de Lima, M., Nascimento, D.C., Schneider, A.H., Caetite, D., Tavares, L.A., Paiva, I.M., et al. (2020). SARS-COV-2-triggered neutrophil extracellular traps mediate COVID-19 pathology. J Exp Med 217, e20201129.
Wang, Y., Li, M., Stadler, S., Correll, S., Li, P., Wang, D., Hayama, R., Leonelli, L., Han, H., Grigoryev, S.A., et al. (2009). Histone hypercitrullination mediates chromatin decondensation and neutrophil extracellular trap formation. J Cell Biol 184, 205-213.
Wong, S.L., Demers, M., Martinod, K., Gallant, M., Wang, Y., Goldfine, A.B., Kahn, C.R., and Wagner, D.D. (2015). Diabetes primes neutrophils to undergo NETosis, which impairs wound healing. Nature medicine 21, 815-819.
Wu, C., Lu, W., Zhang, Y., Zhang, G., Shi, X., Hisada, Y., Grover, S.P., Zhang, X., Li, L., Xiang, B., et al. (2019). Inflammasome Activation Triggers Blood Clotting and Host Death through Pyroptosis. Immunity 50, 1401-1411 e1404.
Xu, J., Zhang, X., Pelayo, R., Monestier, M., Ammollo, C.T., Semeraro, F., Taylor, F.B., Esmon, N.L., Lupu, F., and Esmon, C.T. (2009). Extracellular histones are major mediators of death in sepsis. Nature medicine 15, 1318-1321.
Yang, X., Cheng, X., Tang, Y., Qiu, X., Wang, Y., Kang, H., Wu, J., Wang, Z., Liu, Y., Chen, F., et al. (2019). Bacterial Endotoxin Activates the Coagulation Cascade through Gasdermin D-Dependent Phosphatidylserine Exposure. Immunity 51, 983-996 e986.
Zanoni, I., Tan, Y., Di Gioia, M., Springstead, J.R., and Kagan, J.C. (2017). By Capturing Inflammatory Lipids Released from Dying Cells, the Receptor CD14 Induces Inflammasome-Dependent Phagocyte Hyperactivation. Immunity 47, 697-709.e693.
Zhao, S., Zhang, X., and Li, H. (2018). Beyond histone acetylation—writing and erasing histone acylations. Curr Opin Struct Biol 53, 169-177.
Zhou, F., Yu, T., Du, R., Fan, G., Liu, Y., Liu, Z., Xiang, J., Wang, Y., Song, B., Gu, X., et al. (2020). Clinical course and risk factors for mortality of adult inpatients with COVID-19 in Wuhan, China: a retrospective cohort study. Lancet 395, 1054-1062.
Zuo, Y., Estes, S.K., Ali, R.A., Gandhi, A.A., Yalavarthi, S., Shi, H., Sule, G., Gockman, K., Madison, J.A., Zuo, M., et al. (2020a). Prothrombotic autoantibodies in serum from patients hospitalized with COVID-19. Sci Transl Med, eabd3876.
Zuo, Y., Yalavarthi, S., Shi, H., Gockman, K., Zuo, M., Madison, J.A., Blair, C., Weber, A., Barnes, B.J., Egeblad, M., et al. (2020b). Neutrophil extracellular traps in COVID-19. JCI Insight 5, e138999.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a subject who has a condition associated with NETosis, or reducing the risk of developing or delaying onset of a condition associated with NETosis, the method comprising administering a therapeutically effective amount of an inhibitor of a class I and/or class IIb histone deacetylase (HDAC).

2. The method of claim 1, wherein the condition associated with NETosis is an autoimmune disease, a cardiovascular condition, an inflammatory condition, a condition associated with viral or bacterial infection (optionally bacterial-induced pneumonia or conditions associated with COVID-19 or cytokine storm), or cancer.

3. The method of claim 2, wherein the autoimmune disease is Antineutrophil cytoplasmic antibody (ANCA)-associated vasculitis (AAV), Type 1 diabetes mellitus (TIDM), Systemic lupus erythematosus (SLE), Rheumatoid Arthritis (RA), psoriasis, Antiphospholipid syndrome (APS), multiple sclerosis (MS), dermatomyositis (DM), polymyositis (PM), and IgG4-related autoimmune pancreatitis (AIP), or Drug-Induced Autoimmune Diseases

4. The method of claim 2, wherein the cardiovascular condition is small vessel vasculitis (SVV) or atherosclerosis.

5. The method of claim 2, wherein the inflammatory conditions is gout or Inflammatory bowel diseases (IBDs).

6. A method for treating or reducing the risk of occurrence or reoccurrence, or delaying onset, of a cardiovascular event in a subject who has a condition associated with NETosis, the method comprising administering a therapeutically effective amount of an inhibitor of a class I/IIb histone deacetylase (HDAC).

7. The method of claim 6, wherein the cardiovascular event is thrombosis or myocardial infarction (MI).

8. The method of claim 1, wherein the subject does not have cancer or does not have diabetic peripheral neuropathy.

9. The method of claim 1, wherein the HDAC inhibitor is entinostat, ricolinostat, citarinostat, AES-135; HDAC-IN-3 and analogues thereof; ACY-738; Abexinostat; CAY10603; WT-161; EDO-S101; UF010; Resminostat; HPOB; or CRA-026440.

10. The method of claim 1, wherein the subject has been identified as having a condition associated with NETosis.

11. The method of claim 10, wherein the subject has been identified as having a level of circulating cfDNA, MPO/cfDNA conjugates and/or citH3 above a reference level.

12. The method of claim 10, comprising:
obtaining a sample comprising blood from a subject;
determining a level of circulating cfDNA, MPO/cfDNA conjugates and/or citH3 in the sample;
comparing the level of circulating cfDNA, MPO/cfDNA conjugates and/or citH3 to a reference level that represents a level of circulating cfDNA, MPO/cfDNA conjugates and/or citH3 in a subject who does not have a condition associated with NETosis; and
identifying a subject who has a level of circulating cfDNA, MPO/cfDNA conjugates and/or citH3 above a reference level as having a condition associated with NETosis.

13. The method of claim 1, wherein the subject has been identified as having a perforin mutation, as having a serum ferritin level above a threshold or reference level, or as having a level of a damage-associated molecular pattern (DAMP), optionally high mobility group box-1 (HMGB1), above a threshold or reference level.

14. A composition comprising (i) an anti-viral drug and (ii) a small molecule inhibitor of class I and/or class IIb HDACs.

15. The composition of claim 14, further comprising a pharmaceutically acceptable carrier.

16. The composition of claim 14, wherein the anti-viral drug is nirmatrelvir; ritonavir; sotrovimab; remdesivir; molnupiravir (Lagevrio), or an antibody, optionally bebtelovimab.

17. The composition of 16, wherein the HDAC inhibitor is entinostat, ricolinostat, citarinostat, AES-135; HDAC-IN-3 and analogues thereof; ACY-738; Abexinostat; CAY10603; WT-161; EDO-S101; UF010; Resminostat; HPOB; or CRA-026440

18. A method of treating a viral infection in a subject, the method comprising administering a therapeutically effective amount of the composition of claim 14.

19. The method of claim 18, wherein the viral infection is infection with a coronavirus.

20. The method of claim 19, wherein the coronavirus is SARS-COV-2.

21.-35. (canceled)
